(19) United States
(12) Patent Application Publication
(10) Pub. No.: US 2013/0231307 A1
(43) Pub. Date: Sep. 5, 2013

(54) NOVEL ANTIVIRAL ACYCLIC NUCLEOSIDE PHOSPHONATES

(71) Applicants: Luigi A. Agrofoglio, Orleans (FR); Vincent Roy, Pussay (FR); Hugo Pradere, Lusignan (FR); Jan Balzarini, Heverlee (BE); Robert Snoeck, Sint-Lambrechts-Woluwe (BE); Graciela Andrei, Sint-Lambrechts-Woluwe (BE)

(72) Inventors: Luigi A. Agrofoglio, Orleans (FR); Vincent Roy, Pussay (FR); Hugo Pradere, Lusignan (FR); Jan Balzarini, Heverlee (BE); Robert Snoeck, Sint-Lambrechts-Woluwe (BE); Graciela Andrei, Sint-Lambrechts-Woluwe (BE)

(73) Assignees: CENTRE NATIONAL DE LA RECHERCHE SCIENTIFIQUE, PARIS (FR); KATHOLIEKE UNIVERSITEIT LEUVEN-K.U. LEUVEN R&D, LEUVEN (BE); UNIVERSITE DE ORLEANS, ORLEANS CEDEX 2 (FR)

(21) Appl. No.: 13/822,155

(22) PCT Filed: Jun. 1, 2011
(86) PCT No.: PCT/EP11/59090

§ 371 Date: May 15, 2013

(30) Foreign Application Priority Data
Sep. 14, 2010 (EP) 10305983.8

(51) Int. Cl. A61K 31/675; C07F 9/6561; A61K 45/06; C07F 9/6512

(52) U.S. Cl. CPC A61K 31/675; C07F 9/65121; C07F 9/65616; A61K 45/06
USPC 514/81; 544/243; 514/86; 544/244

(57) ABSTRACT

Compounds of formula (I) wherein A represents a nucleobase or a derivative thereof, n is equal to 0 or 1 and R′, R″ are carbonic chain, and R₁₀ is hydrogen or a carbonic chain for their use as antiviral agents.

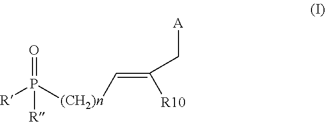

DNA viruses and retroviruses cause severe diseases spreading among the world population. Current therapies (i.e. HIV treatment) often need permanent drug administration to maintain low or undetectable viral levels in patients. However drug-resistant viruses frequently appear, so new antiviral derivatives are still needed. In the last ten years, acyclic nucleoside phosphonates (ANPs) like adefovir (9-(2-Phosphonylmethoxyethyl)-adenine or PMEA), tenofovir [(1R)-9-(2-Phosphonylmethoxypropyl)-adenine or (R)-PMPA] and cidofovir [(S)-CDV] became major antiviral nucleotide derivatives that are approved for the treatment of severe virus infections. They mimic natural nucleotides (dAMP or dCMP) and thus bypass the first phosphorylation step by a nucleoside kinase. Their activities depend on metabolic conversion by two salvage pathway kinases (NMP and NDP kinases) to their diphosphates (ANP-PPs), followed by a specific interaction with the virus DNA polymerase. ANP-PPs, like the other antiviral nucleotide analogs compete with the endogenous dNTP pools for binding to the virus DNA polymerase and incorporation into the growing virus DNA chain, inhibiting viral (i.e. in HIV or vaccinia) replication. ANPs and their phosphorylated forms are resistant to nucleotidases and have long half-lifes in the body, for example >15H for PMEA-PP. However antiviral treatment involving nucleoside or nucleotide analogs frequently result in the selection of resistant virus strains, with precise point mutations in the DNA polymerase gene. The only acyclic pyrimidine nucleoside phosphonate analogs found to be effective to date is cidofovir (CDV) and the acyclic 2,4-diaminopyrimidine nucleoside phosphonates (i.e. PMEO-DAPym); Balzarini, J et al. Antimicrob. Agents Chemother. 2002, 46, 2185-93; Hocková, D. et al. J. Med. Chem. 2003, 46, 5064-5073.

CDV has a broad antiviral activity and has been approved by the FDA for use against cytomegalovirus infections. The PMEO-DAPym derivatives are currently not subject of clinical trials. The first phosphorylation step of antiviral nucleoside analogs is usually the rate-determining step in the salvage pathway. In a similar way, the efficacy of ANPs should depend on their intracellular phosphorylation and the amounts of their active form, ANP-PP. All studied ANPs are slowly phosphorylated by human NMP kinases: AMP kinases 1 and 2 for PMEA and (R)-PMPA and UMP-CMP kinase (hUCK) for cidofovir. The last phosphorylation step is performed by several enzymes, including NDP kinases and creatine kinases.

WO 2006/137953 discloses only phosphono-Pent-2-En-1-yl Nucleosides under a trans and cis-forms which are useful as antiviral agents. In these compounds the phosphonopentenyl group corresponds to the acyclic sugar moiety and said phosphonopentenyl side chain design was chosen so that the resulting analogs would resemble the corresponding portion of 2′,3′-didehydro-2′,3′-dideoxynucleosides, such as stavudine, 2′,3′-didehydro-2′,3′-dideoxyadenosine, and abacavir. Said compounds mimic the C1′, C2′, C3′, C4′ and C5′ bounds of the ribofuranose moiety or analogs especially with the cis double bound such the one found in abacavir.

The inventors have synthesised several [E]-3-(N¹-uracil)-propenyl phosphonic acid corresponding to formula (6a-e)

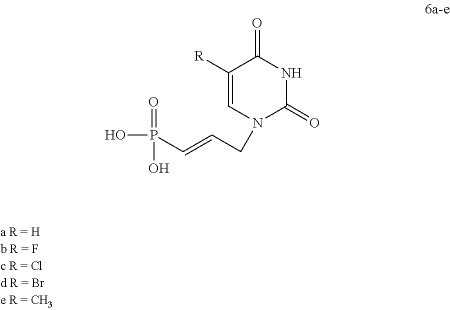

which were found with affinities for nucleotide kinases that are similar to dUMP and dCMP, the natural substrates, and higher than that of cidofovir which is not a substrate for hUCK (Topalis, D et al. FEBS J. 2007, 274, 3704-14).

But, there is a need for compounds which could be efficient substrates for hUCK, potentially resulting in better antiviral activities.

In course of their work, the inventors have defined the structural requirements for intracellular activation leading to new compounds with better bioavalaibility. They found that by mimicking the chaining of C1′, O, C4′ and C5′ of a ribofuranose structure, the length of the alkyl chain may be reduced. The double bound is preferably in the trans form.

Thus an object of the instant invention is compounds of formula (I)

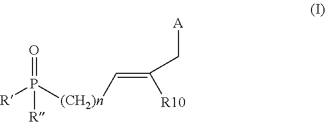

wherein
A represents a nucleobase or a derivative thereof,
n is equal to 0 or 1 and
R′ and R″ independently of each other
   * represent a group selected from the group comprising:
      a hydroxy group, with the proviso that R′ and R″ are not simultaneously a hydroxy group,
      O-methyl group,
      O-benzyl group,
      an oxymethylcarbonyl group of formula (2)

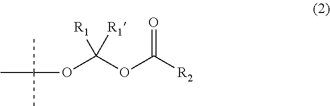

wherein
R₁ and R′₁ are independently of each other hydrogen or (C₁-C₄)alkyl group and
R₂ is a straight or branched (C₁-C₆)alkyl group or straight or branched (C₁-C₆)alkoxy group
a thioethylcarbonyl group of formula (3)

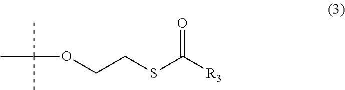

wherein
R₃ is a straight or branched (C₁-C₆)alkyl group
a lipohilic chain or
* R′ and R″ forms with the phosphate atom to which they are linked a cycloalkyle group of formula (4)

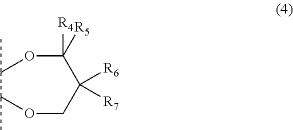

wherein R₄, R₅, R₆, et R₇ each independently represent a straight or branched (C₁-C₆)alkyl group or R₄, and R₇ independently represent a straight or branched (C₁-C₆)alkyl group and R₅ and R₆ form together an aromatic ring,
R₁₀ represents a hydrogen atom or a straight or branched (C₁-C₄)alkyl group optionally substituted by an OH group,
   their diastereoisomers, or a pharmaceutically acceptable salt thereof,
   for their use as antiviral agents, for the treatment of virus pathologies.

According to the invention the term nucleobase stands for natural or non natural purines and pyrimidines bases selected from, but not limited to, the group comprising uracil, thymine, cytosine, guanine, purine, hypoxanthine and adenine and analogs thereof. The term analogs thereof stands for natural derivatives or synthetic derivatives of said nucleobases such as but not limited to dihydrouridine, inosine, hypoxanthine and xanthine or nucleobase optionally substituted by an halogen atom, a (C₁-C₆)alkyl group or an aryl group, like for example theophylline, theobromine and caffeine.

The term halogen stands for fluorine, chlorine, bromine and iodine.

The term straight or branched (C₁-C₆)alkyl group stands for a straight-chain or branched hydrocarbon residue containing 1-6 C-atoms, such as, methyl, ethyl, propyl, isopropyl, n-butyl, isobutyl, t-butyl, n-pentyl, isopentyl, n-hexyl.

The term (C₁-C₆)alkoxy group stands for alkyl-O— with alkyl as defined above, e.g. methoxy, ethoxy, n-propoxy, iso-propoxy, n-butoxy, isobutoxy, t-butoxy, n-pentoxy, isopentoxy and hexoxy.

The term lipophilic chain or long-chain refers to the cyclic, branched or straight chain chemical groups that when covalently linked to a phosphonic acid to form a phosphonate ester increase oral bioavailability and enhance activity as for instance for some nucleoside phosphonates when compared with the parent nucleoside. These lipophilic groups include, but are not limited to, aryl, alkyl, alkoxyalkyl, and alkylglyceryl (such as hexadecyloxypropyl (HDP)-, octadecyloxyethyl-, oleyloxypropyl-, and oleyloxyethyl-esters).

The term aromatic ring stands for, but is not limited to, aryl, e.g. phenyl, benzyl, naphtyl or indanyl, said aryl group being optionally substituted.

As used herein, pharmaceutically acceptable derivatives of the compounds according to the present invention include salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization. The compounds produced may be administered to animals or humans without substantial toxic effects and either are pharmaceutically active or are prodrugs.

Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethyl-benzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, nitrates, borates, methanesulfonates, benzenesulfonates, toluenesulfonates, salts of mineral acids, such as but not limited to hydrochlorides, hydrobromides, hydroiodides and sulfates; and salts of organic acids, such as but not limited to acetates, trifluoroacetates, maleates, oxalates, lactates, malates, tartrates, citrates, benzoates, salicylates, ascorbates, succinates, butyrates, valerates and fumarates. Pharmaceutically acceptable esters include, but are not limited to, alkyl, alkenyl, alkynyl, and cycloalkyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is hydrogen, alkyl, alkenyl, alkynyl, and cycloalkyl. Pharmaceutically acceptable enol esters include, but are not limited to, derivatives of formula C═C(OC(O)R) where R is hydrogen, alkyl, alkenyl, alkynyl, or cycloalkyl. Pharmaceutically acceptable solvates and hydrates are complexes of a compound with one or more solvent or water molecules, or 1 to about 100, or 1 to about 10, or one to about 2, 3 or 4, solvent or water molecules.

According to the invention the compounds of formula (I) may be used in the treatment of virus pathologies, for example, but not limited to, pathologies due to Herpes virus, Vaccinia virus, Varicella-zoster virus, Cytomegalovirus, Vesicular stomatis virus, Influenza A, Coxsackie virus B4, respiratory syncytial virus, Feline corona virus, Feline herpes virus, Punta Toro Virus, HIV, Hepatitis B and Hepatitis C.

In an advantageous embodiment according to the invention the Varicella-zoster virus does not necessarily need to show any encoded thymidine kinase activity (so called VZV−TK⁽⁻⁾)

Another object of the invention is compounds of formula (I) wherein A is selected from a natural or non natural pyrimidine and purine bases said base being selected from the group comprising:

a)

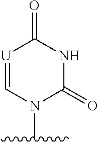

wherein U represents a nitrogen atom or C—R₁₁ with R₁₁ selected from the group comprising
a hydrogen atom,
an halogen atom selected from fluorine, chlorine, bromine and iodine,
a straight or branched (C₁-C₆)alkyl group as defined above, advantageously a methyl or isopropyl group,
a

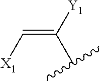

group with X₁
   representing a halogen atom as defined above, preferably iodine, bromine or chlorine and Y₁ representing hydrogen atom or a halogen atom as defined above, preferably iodine, bromine or chlorine,
a phenyl group optionally substituted in the 4 position by a (C₁-C₆)alkyl group as defined above, a (C₁-C₆)alcoxy group as defined above, a phenyloxy, a 3,4-ethylenedioxy or a 3,4-methyledioxy, preferably a butyl group
a group selected from the group comprising thiophenyl, pyrrolyl or furanyl groups and their derivatives,

b)

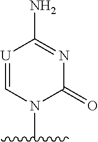

wherein U represents a nitrogen atom or C—R₁₂ with R₁₂ selected from the group comprising
a hydrogen atom, or
an halogen atom selected from fluorine, chlorine, bromine and iodine,

c)

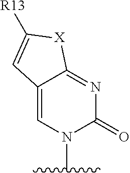

wherein X represents an oxygen or a sulphur atom and R₁₃ represents a group selected from

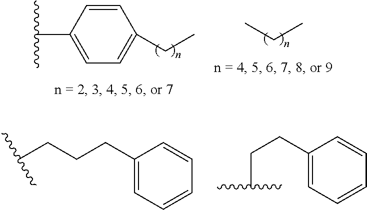

d)

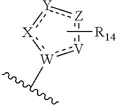

wherein

V, W, X, Y, Z represents each independently of the other C or N, and in particular wherein W, X and Y or W, Y and V or W and Y are N
[symbol] means a single or double bond according to the meaning of V, W, X, Y, Z and
R₁₄ is selected from the group comprising a hydrogen atom, a halogen atom, (C1-C4)alkyl groups, a phenyl group, ester groups, amide groups,

e)

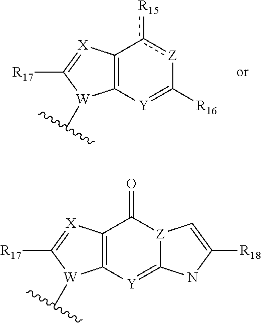

wherein

W, X, Y, Z represents each independently of the other C or N,

[symbol] means a single or double bond according to the meaning of W, X, Y, Z and R₁₅,

R₁₅ is selected from the group comprising
an halogen atom as defined above, advantageously a chlorine or a bromine,
an oxygen atom,
an amino group, or
a group selected from

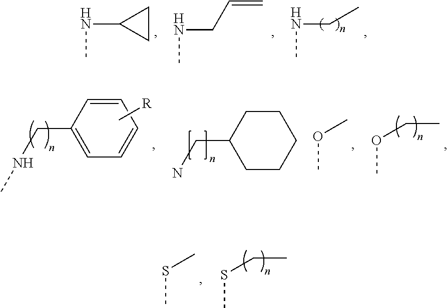

groups with R representing a straight or branched (C₁-C₄)alkyl group, preferably an isopropyl group and n being an integer equal to 0, 1, 2 or 3,

R₁₆ is a hydrogen atom or an amino group,

R₁₇ is selected from the group comprising a hydrogen atom, a halogen atom, (C₁-C₄)alkyl group and a phenyl group and

R₁₈ is selected from the group comprising a hydrogen atom, a halogen atom, (C1-C4)alkyl groups, ester groups and aromatic groups for their use as antiviral agents, for the treatment of virus pathologies.

Another object of the invention are compounds of formula (I) wherein R′ and R″ independently from each other are selected from the group comprising O-methyl, O-benzyl, or a bio labile group such as an oxymethylcarbonyl group (such as OPOM, OPOC) or an alcoxyalkyl ester prodrugs (such as OHDP), for their use as antiviral agents, for the treatment of virus pathologies. POM states for pivaloyl oxymethyl [(CH₃)₃C—CO—O—CH₂—], POC for isopropyloxymethylcarbonate [(CH₃)₂—C—O—C(O)—O—CH₂—], and HDP for hexadecyloxypropyl [CH₃ (CH₂)₁₅—O—(CH₂)₃—].

Another object of the invention is compounds of formula (I) wherein n is equal to 1, for their use as antiviral agents, for the treatment of virus pathologies.

Another object of the invention are compounds of formula (I) wherein:

R₁₀ is a hydrogen atom,

n is equal to 1,

R′ and R″ represent each independently OH, OPOC, OPOM and OHDP, with the proviso that they are never simultaneously OH, and

A is as defined above.

In a preferred embodiment of the invention, the compounds are those of formula (I′).

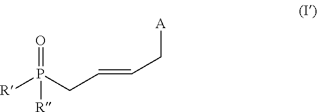

wherein
R′ and R″ are independently of each other selected from the group comprising OH, OPOC, OPOM and OHDP, with the proviso that they are never simultaneously OH, and
A is selected from the group comprising:
   a)

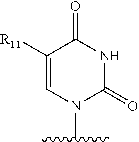

with R₁₁ being H, F, Cl, Br, CH₃ or a group

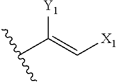

with X═Y═I or X═Y═Br or X═I and Y═Br ou Cl
   b)

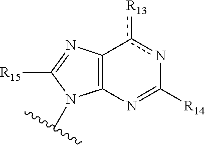

with R₁₃ selected from
O, Cl, OCH₃, NH₂ and a group

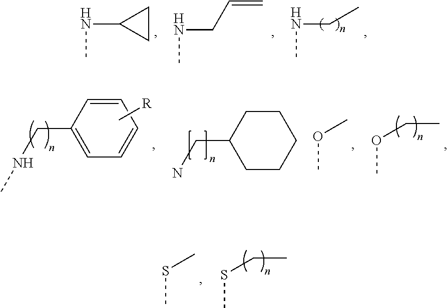

with R representing a straight or branched (C₁-C₄)alkyl group, preferably an isopropyl group and n being an integer equal to 0, 1, 2 or 3,
R₁₅ being H and R₁₄ being H or NH₂,
c)

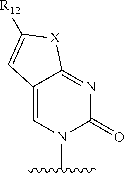

wherein X represents an oxygen atom and R₁₂ represents a group selected from

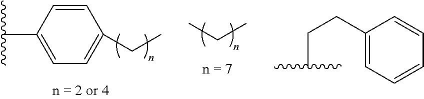

Another object of the invention is compounds of formula (I) wherein R₁₀ is H or of formula R′, which are under the E form, for their use as antiviral agents, for the treatment of virus pathologies.

for their use as antiviral agents, for the treatment of virus pathologies.

Another object of the invention is compounds of formula (I) selected from the group comprising:

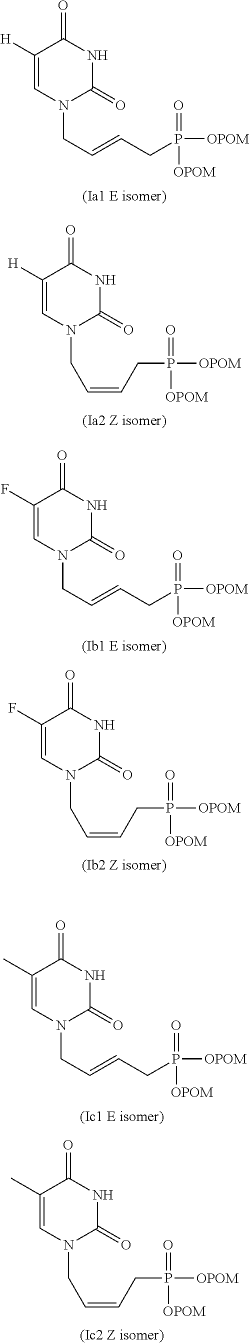

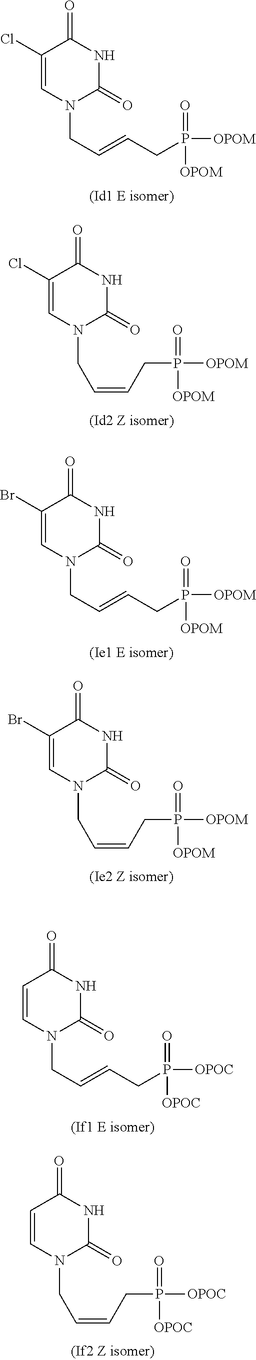

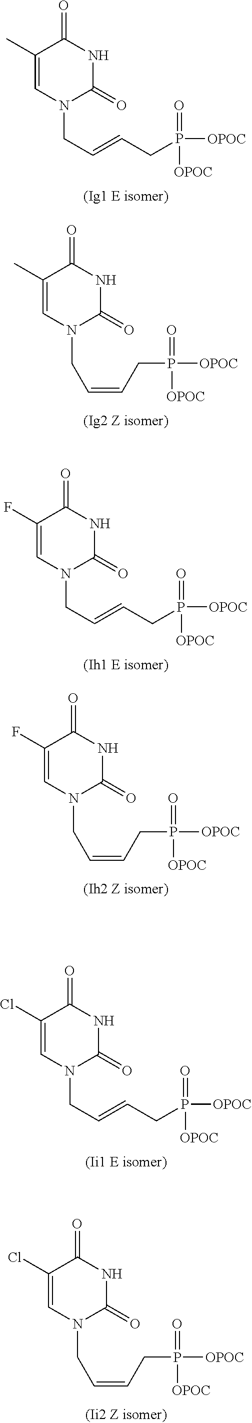

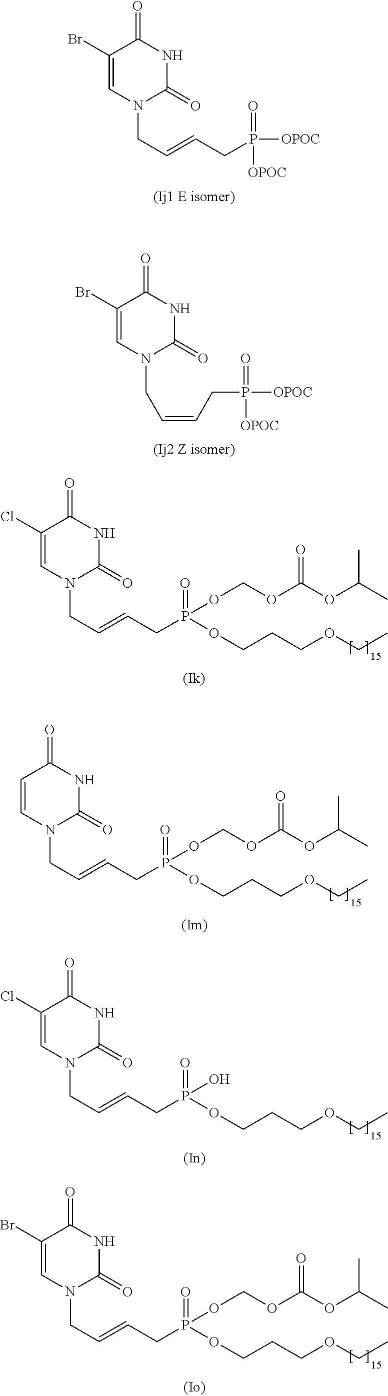

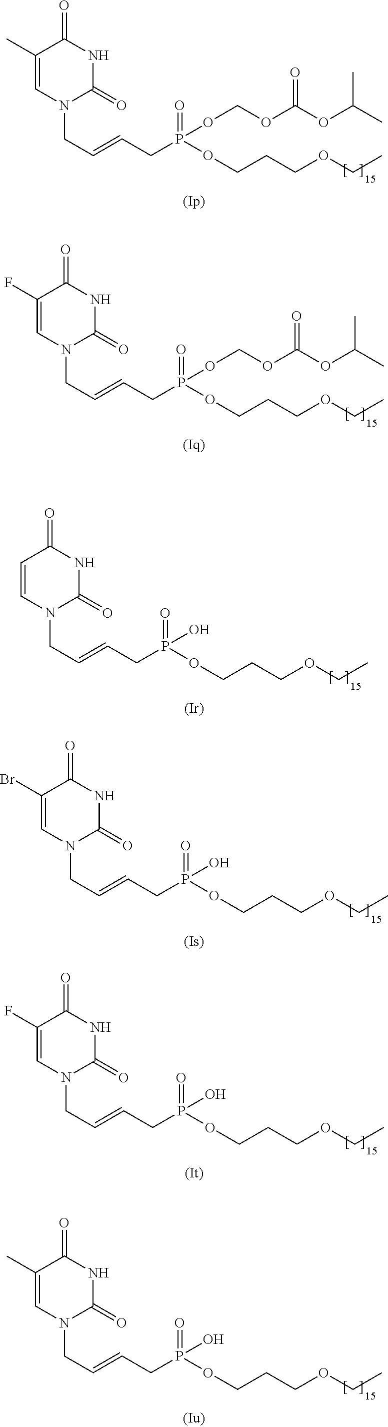

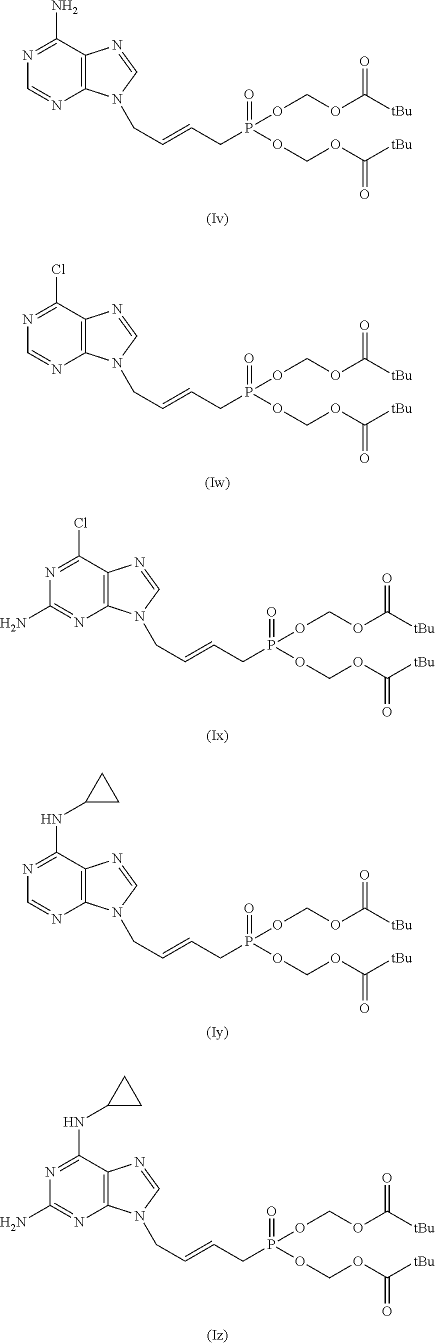 [SMILES: CC(C)(C)C(=O)OCOP(=O)(C/C=C/Cn1cnc2c(N)ncnc21)OCOC(=O)C(C)(C)C ; CC(C)(C)C(=O)OCOP(=O)(C/C=C/Cn1cnc2c(Cl)ncnc21)OCOC(=O)C(C)(C)C ; CC(C)(C)C(=O)OCOP(=O)(C/C=C/Cn1cnc2c(Cl)nc(N)nc21)OCOC(=O)C(C)(C)C ; CC(C)(C)C(=O)OCOP(=O)(C/C=C/Cn1cnc2c(NC3CC3)ncnc21)OCOC(=O)C(C)(C)C ; CC(C)(C)C(=O)OCOP(=O)(C/C=C/Cn1cnc2c(NC3CC3)nc(N)nc21)OCOC(=O)C(C)(C)C]

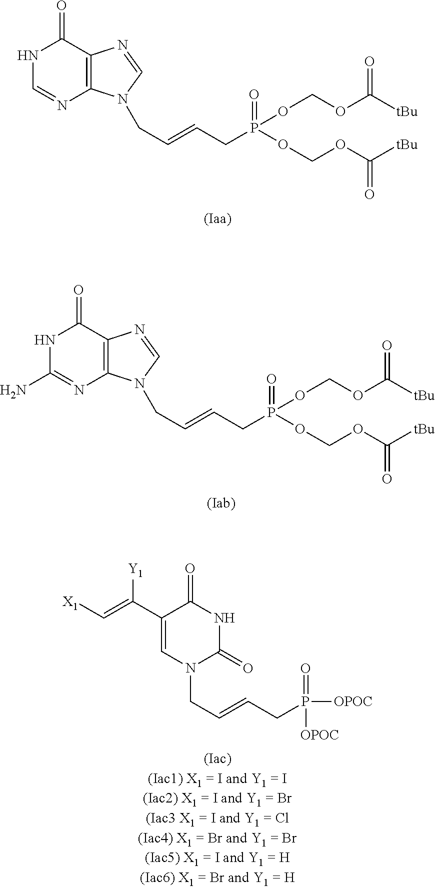

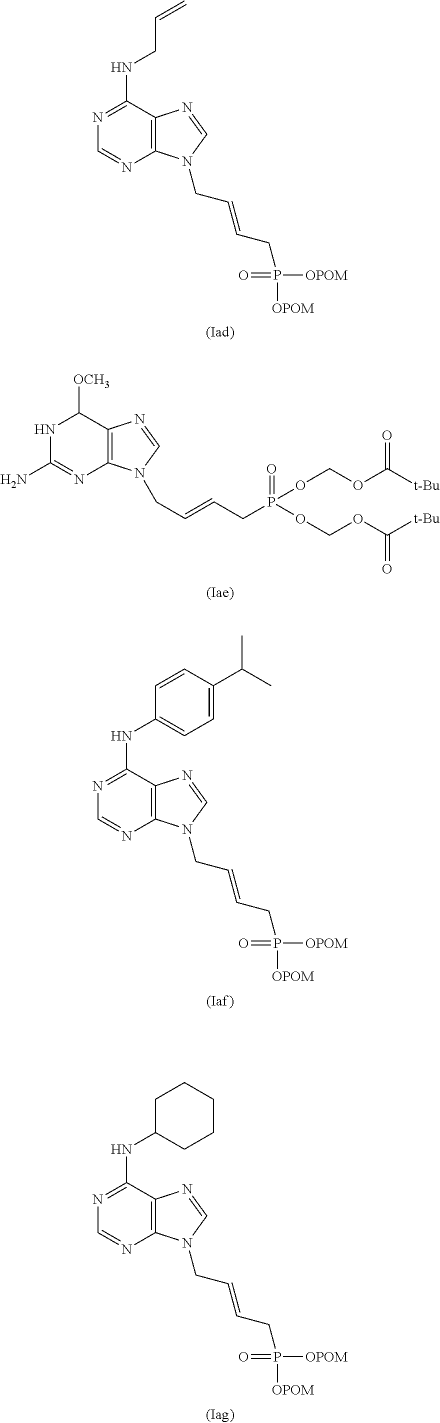

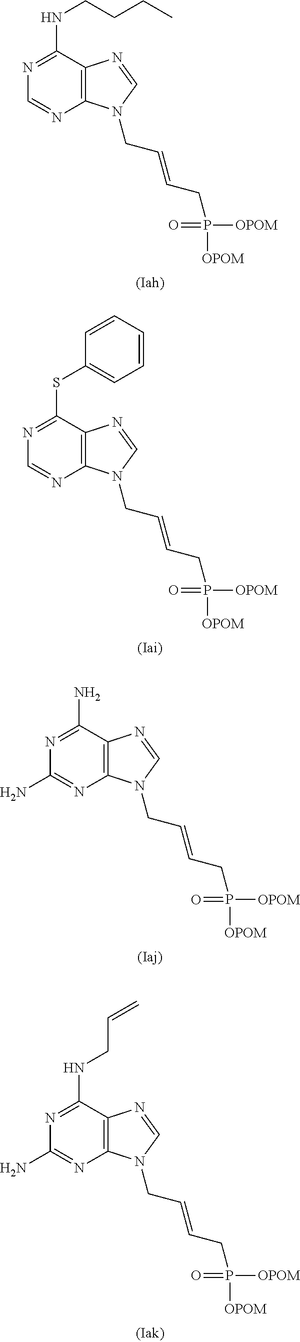

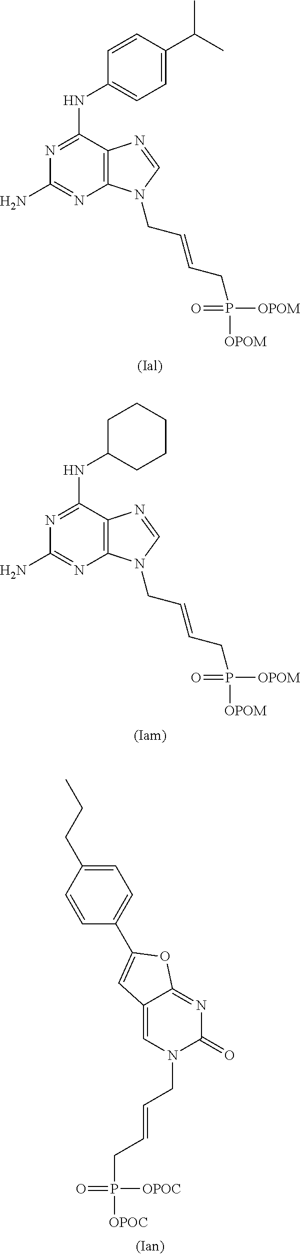

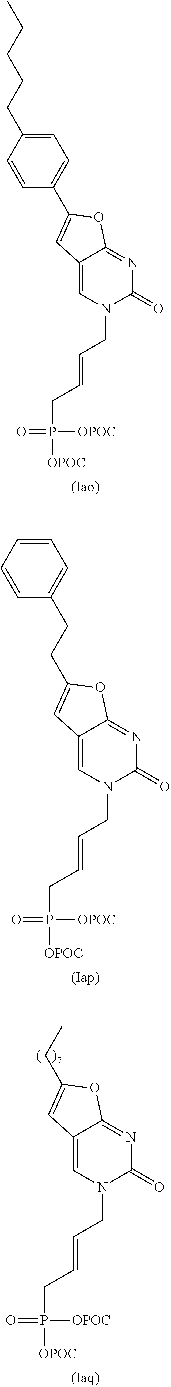

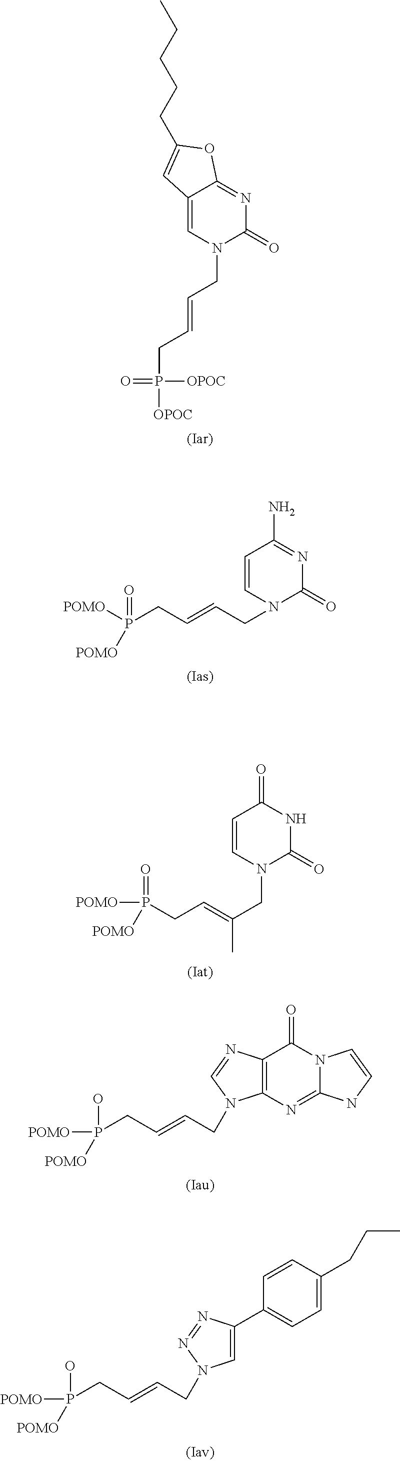

for their use as antiviral agent, for the treatment of virus pathologies.

The compounds used according to the invention may be prepared by any methods known in the art or described in the literature from compounds disclosed in the literature or commercially available.

For example uracil phosphonates derivatives may be prepared according to procedures previously described (Kumamoto, H.; Broggi, J.; Topalis, D.; Pradere, U.; Roy, V.; Berteina-Raboin, S.; Nolan, S. P.; Deville-Bonne, D.; Snoeck, R.; Garin, D.; Agrofoglio, L. Preparation of acyclo nucleoside phosphonate analogues based-on cross-metathesis. Tetrahedron 2008, 64, 3517-3526; Roy, V.; Kumamoto, H.; Berteina-Raboin, S.; Nolan, S. P.; Topalis, D.; Deville-Bonne, D.; Balzarini, J.; Neyts, J.; Andrei, G.; Snoeck, R.; Agrofoglio, L. A. Cross-metathesis mediated synthesis of new acyclic nucleoside phosphonates. Nucleosides Nucleotides Nucleic Acids 2007, 26, 1399-1402).

Another object of the invention is pharmaceutical compositions comprising at least one compound of formula (I) in association with a pharmaceutically acceptable carrier. Pharmaceutical carriers suitable for administration of the compounds provided herein include any such carriers known to those skilled in the art to be suitable for the particular mode of administration.

According to the invention, the pharmaceutical composition may further comprise an other active ingredients like for example antiviral compounds selected from, but not limited to, the group comprising Abacavir, Acyclovir, Adefovir, Amprenavir, Atazanavir, Cidofovir, Darunavir, Delavirdine, Didanosine, Docosanol, Éfavirenz, Elvitégravir, Emtricitabine, Enfuvirtide, Étravirine, Famciclovir, Foscarnet, Fomivirsen, Ganciclovir, Indinavir, Idoxuridine, Lamivudine, Lopinavir Maraviroc, MK-2048, Nelfinavir, Névirapine, Penciclovir, Raltégravir, Rilpivirine, Ritonavir, Saquinavir, Stavudine, Ténofovir Trifluridine, Valaciclovir, Valganciclovir, Vidarabine, Ibacitabine, Amantadine, Oseltamivir, Rimantidine, Tipranavir, Zalcitabine, Zanamivir et Zidovudine.

The pharmaceutical compositions contain one or more compounds and the compounds may be formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. All the techniques and procedures used are well known in the art.

The active compound is included in the pharmaceutically acceptable carrier in an amount sufficient to exert a therapeutically useful effect in the absence of undesirable side effects on the patient treated. The therapeutically effective concentration may be determined empirically by testing the compounds in vitro and in vivo systems well known to those of skill in the art and then extrapolated there from for dosages for humans.

The concentration of active compound in the pharmaceutical composition will depend on absorption, inactivation and excretion rates of the active compound, the physicochemical characteristics of the compound, the dosage schedule, and amount administered as well as other factors known to those of skill in the art.

In one embodiment, a therapeutically effective dosage should produce a serum concentration of active ingredient of from about 0.1 ng/ml to about 50-100 μg/ml. The pharmaceutical compositions, in another embodiment, should provide a dosage of from about 0.001 mg to about 2000 mg of compound per kilogram of body weight per day. Pharmaceutical dosage unit forms are prepared to provide from about 0.01 mg, 0.1 mg or 1 mg to about 500 mg, 1000 mg or 2000 mg, and in one embodiment from about 10 mg to about 500 mg of the active ingredient or a combination of essential ingredients per dosage unit form. The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Another object according to the invention is pharmaceutical compositions comprising at least one compound of formula (I) for their use as antiviral agent.

Still another object of the invention is the use of at least one compound of formula (I) for the preparation of a medicament useful as antiviral agent.

Finally another object of the invention is a method for treating virus pathologies, for example, but not limited to, pathologies due to Herpes virus, Vaccinia virus, Varicella-zoster virus, Cytomegalovirus, Vesicular stomatis virus, Influenza A, Coxsackie virus B4, respiratory syncytial virus, Feline corona virus, Feline herpes virus, Punta Toro Virus, HIV, Hepatitis B and Hepatitis C, said method comprising a step of administering in a patient in need thereof a therapeutically effective amount of at least one compound of formula (I).

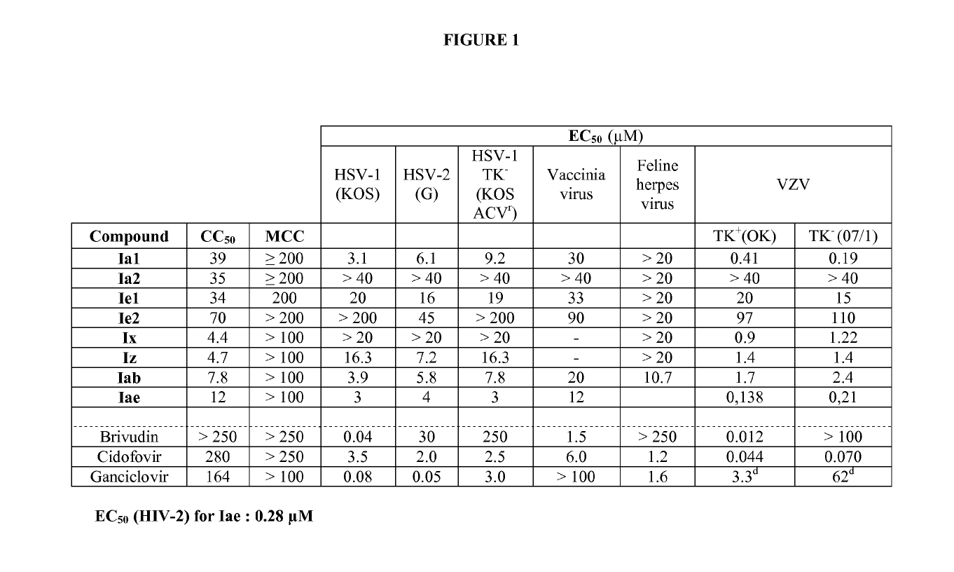

The examples 1 to 4 and FIG. 1 illustrate the invention.

FIG. 1 illustrates the antiviral activities and cytotoxicities of the compounds according to the invention in HEL cell cultures as measured according to example 3. CE₅₀ represents the effective concentration (μM), required to reduce virus-induced cytopathogenicity by 50%. CC₅₀ represents the cytostatic concentration (μM), required to inhibit HEL cell proliferation by 50%. MMC represents the minimum cytotoxic concentration (μM), required to cause a microscopically detectable alteration of normal cell morphology. Birvudin, Cidofovir and Gamciclovir are reference compounds. TK⁺ Varicella-zoster virus encoding thymidine kinase activity; TK⁻ Varicella-zoster virus not encoding thymidine kinase activity.

### EXAMPLE 1

### Synthesis of Uracil Phosphonates Derivatives: General Procedure

The synthesis is illustrated in the following scheme

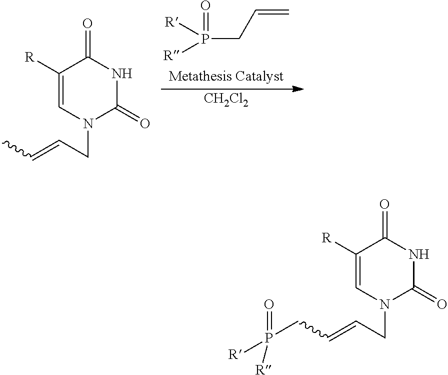

To a CH₂Cl₂ (25 mL/mmol) solution of N¹-crotyl-5-substituted uracil (1 equiv.) and bis(POM), bis(POC) or (HDP/POC) allylphosphonate (1.3 equiv.) was added metathesis catalyst (0.05 equiv.) then this solution was stirred under positive pressure of dry air. After evaporation of all volatiles, the residue was purified by silica gel column chromatography to give the desired products.

According to said procedure, the following compounds are obtained and characterised

### 1.1. (E)-N¹-(4′-bis(POM)-phosphinyl-2′-butenyl)uracil (Ia1)

IR: 2977, 1751, 1685, 1459, 1242, 1138, 956, 855 cm⁻¹.

¹H NMR (400 MHz, CDCl₃) δ 8.73 (s, 1H, NH), 7.17 (d, J=7.9 Hz, 1H, H⁶), 5.75-5.61 (m, 7H, O—CH₂—O, H^{2′}, H^{3′}, H⁵), 4.32 (t, J=4.1 Hz, 2H, H^{1′}), 2.72 (dd, J=22.4, 5.0 Hz, 2H, H^{4′}), 1.23 (s, 18H, tBu).

¹³C NMR (100 MHz, CDCl₃) δ 176.8 (C═O), 163.2 (C═O), 150.5 (C═O), 143.4 (C⁶), 129.5, 129.3 (C²), 124.1, 124.0 (C^{3′}), 102.6 (C⁵), 81.6, 81.5 (O—CH₂—O), 49.1, 49.0 (C^{1′}), 38.7 (C(CH₃)₃), 31.5, 30.1 (C^{4′}), 26.8 (C(CH₃)₃).

³¹P NMR (162 MHz, CDCl₃) δ 26.4.

HRMS (ESI): m/z [M+Na]⁺ calcd for C20H31N2NaO9P: 497.1665, found: 497.1674.

1.2. (Z)—N¹-(4′-bis(POM)-phosphinyl-2′-butenyOuracil (Ia2)

¹H NMR (400 MHz, CDCl₃) δ 8.32 (s, 1H, NH), 7.47 (d, J=7.9 Hz, 1H, H⁶), 5.77-5.60 (m, 7H, O—CH₂—O, H^{2′}, H^{3′}, H⁵), 4.44 (t, J=4.7 Hz, 2H, H″), 2.82 (dd, J=23.4, 6.7 Hz, 2H, H^{4′}), 1.24 (s, 18H, tBu).

¹³C NMR (100 MHz, CDCl₃) δ 177.0 (C═O), 163.2 (C═O), 150.6 (C═O), 144.4 (C⁶), 129.1, 128.9 (C^{2′}), 122.1, 122.0 (C^{3′}), 102.5 (C⁵), 81.6, 81.5 (O—CH₂—O), 44.8, 44.7 (C^{1′}), 38.8 (C(CH₃)₃), 27.0 and 25.6 (C^{4′}), 26.9 (C(CH₃)₃).

³¹P NMR (162 MHz, CDCl₃) δ 26.6.

HRMS (ESI): m/z [M+Na]⁺ calcd for C20H31N2NaO9P: 497.1665, found: 497.1677.

### 1.3. (E)-N¹-(4′-bis(POM)-phosphinyl-2′-butenyl)-5-fluoro-uracil (Ib1)

IR: 2977, 2361, 1752, 1702, 1480, 1238, 1137, 965, 871 cm⁻¹.

¹H NMR (400 MHz, CDCl₃) δ 9.58 (s, 1H, NH), 7.28 (d, J=5.5 Hz, 1H, H⁶), 5.77-5.61 (m, 6H, O—CH₂—O, H^{2′}, H^{Y}), 4.31 (t, J=4.9 Hz, 2H, H″), 2.72 (dd, J=22.6, 5.3 Hz, 2H, H^{4′}), 1.22 (s, 18H, tBu).

¹³C NMR (100 MHz, CDCl₃) δ 176.8 (C═O), 157.2, 156.9 (C═O), 149.3 (C═O), 141.7, 139.3 (C⁵), 129.1, 128.9 (C^{2′}), 127.7, 127.4 (C⁶), 124.7, 124.6 (C^{3′}), 81.6, 81.5 (O—CH₂—O), 49.3 (2C, C^{1′}), 38.6 (C(CH₃)₃), 31.4, 30.0 (C^{4′}), 26.7 (C(CH₃)₃).

³¹P NMR (162 MHz, CDCl₃) δ 26.3.

HRMS (ESI): m/z [M+Na]⁺ calcd for C20H30N2NaO9FP: 515.1571, found: 515.1579.

### 1.4. (Z)—N¹-(4′-bis(POM)-phosphinyl-2′-butenyl)-5-fluoro-uracil (Ib2)

¹H NMR (400 MHz, CDCl₃) δ 8.59 (s, 1H, NH), 7.73 (d, J=5.8 Hz, 1H, H⁶), 5.75-5.61 (m, 6H, O—CH₂—O, H^{2′}, H^{3′}), 4.44 (t, J=4.7 Hz, 2H, H″), 2.80 (dd, J=23.5, 6.8 Hz, 2H, H^{4′}), 1.23 (s, 18H, tBu).

¹³C NMR (100 MHz, CDCl₃) δ 177.0 (C═O), 157.0, 156.7 (C═O), 149.2 (C═O), 141.7, 139.4 (C⁵), 128.9, 128.7, 128.6 (2C, C^{2′}, and C⁶), 122.6, 122.5 (C^{3′}), 81.7, 81.6 (O—CH₂—O), 44.9 (2C, C^{1′}), 38.8 (C(CH₃)₃), 26.9 and 25.5 (C^{4′}), 26.8 (C(CH₃)₃).

³¹P NMR (162 MHz, CDCl₃ δ 26.4.

HRMS (ESI): m/z [M+Na]⁺ calcd for C20H30N2NaO9FP: 515.1571, found: 515.1584.

### 1.5. (E)-N¹-(4′-bis(POM)-phosphinyl-2′-butenyl)thymine (Ic1)

IR: 2929, 1678, 1453, 1396, 1196, 986, 751 cm⁻¹.

¹H NMR (400 MHz, CD₃OD) δ 7.37 (d, J=0.9 Hz, 1H, H⁶), 5.85-5.74 (ttd, J=15.4, 11.2, 5.1 Hz, H^{2′}), 5.66 (m, 5H, O—CH₂—O, H^{3′}), 4.32 (t, J=5.2 Hz, 2H, H″), 2.82 (dd, J=22.5, 7.2 Hz, 2H, H^{4′}), 1.87 (s, 3H, CH₃—U) 1.23 (s, 18H, tBu).

¹³C NMR (100 MHz, CDCl₃) δ 178.1 (C═O), 166.8 (C═O), 152.7 (C═O), 142.4 (C⁶), 131.8, 131.6 (C^{2′}), 123.7, 123.6 (C^{3′}), 111.5 (C⁵), 83.2, 83.1 (O—CH₂—O), 49.9 (2C, C^{1′}), 39.7 (C(CH₃)₃), 31.8, 30.4 (C^{4′}), 27.2 (C(CH₃)₃), 12.3 (CH₃—U).

³¹P NMR (162 MHz, CD₃OD) δ 27.3.

HRMS (ESI): m/z [M+Na]⁺ calcd for C21H33N2NaO9P: 511.1821, found: 511.1837.

### 1.6. (Z)—N¹-(4′-bis(POM)-phosphinyl-2′-butenyl)thymine (Ic2)

¹H NMR (400 MHz, CD₃OD) δ 7.46 (d, J=1.2 Hz, 1H, H⁶), 5.78-5.60 (m, 6H, O—CH₂—O, H^{2′}, H^{3′}), 4.40 (dd, J=5.5, 3.8 Hz, 2H, H^{1′}), 3.01 (dd, J=23.2, 7.8 Hz, 2H, H^{4′}), 1.88 (d, J=1.1 Hz, 3H, CH₃—U), 1.24 (d, J=3.2 Hz, 18H, tBu).

¹³C NMR (100 MHz, CD₃OD) δ 178.2 (C═O), 166.9 (C═O), 152.9 (C═O), 142.7 (C⁶), 130.6, 130.4 (C^{2′}), 123.0, 122.8 (C^{3′}), 111.5 (C⁵), 83.2, 83.1 (O—CH₂—O), 45.6 (C^{1′}), 39.8 (C(CH₃)₃), 27.7 and 26.3 (C^{4′}), 27.3 (C(CH₃)₃), 12.3 (CH₃—U).

³¹P NMR (162 MHz, CD₃OD) δ 27.6.

HRMS (ESI): m/z [M+Na]⁺ calcd for C₂₁H₃₃N₂NaO9P: 511.1821, found: 511.1835.

### 1.7. (E)-N¹-(4′-bis(POM)-phosphinyl-2′-butenyl)-5-chloro-uracil (Id1)

IR: 2976, 1752, 1692, 1452, 1236, 1135, 963, 853 cm⁻¹.

¹H NMR (400 MHz, CDCl₃) δ 8.73 (s, 1H, NH), 7.42 (s, 1H, H⁶), 5.80-5.60 (m, 6H, O—CH₂—O, H^{2′}, H^{3′}), 4.33 (t, J=4.5 Hz, 2H, H^{1′}), 2.72 (dd, J=22.6, 5.7 Hz, 2H, H^{4′}), 1.23 (s, 18H, tBu).

¹³C NMR (100 MHz, CDCl₃) δ 176.8 (C═O), 158.8 (C═O), 149.4 (C═O), 140.3 (C⁶), 129.0, 128.8 (C^{2′}), 125.0, 124.9 (C^{3′}), 109.0 (C⁵), 81.6 (2C, O—CH₂—O), 49.5 (2C, C^{1′}), 38.7 (C(CH₃)₃), 31.5, 30.1 (C^{4′}), 26.8 (C(CH₃)₃).

³¹P NMR (162 MHz, CDCl₃) δ 26.1.

HRMS (ESI): m/z [M+Na]⁺ calcd for C20H30N2NaO9ClP: 531.1275, found: 531.1293.

### 1.8. (Z)—N¹-(4′-bis(POM)-phosphinyl-2′-butenyl)-5-chloro-uracil (Id2)

¹H NMR (400 MHz, CDCl₃) δ 8.48 (s, 1H, NH), 7.79 (s, 1H, H⁶), 5.74-5.62 (m, 6H, O—CH₂—O, H^{2′}, H^{3′}), 4.47 (t, J=4.6 Hz, 2H, H″), 2.82 (dd, J=23.5, 6.9 Hz, 2H, H^{4′}), 1.24 (s, 18H).

¹³C NMR (100 MHz, CDCl₃) δ 177.0 (C═O), 158.9 (C═O), 149.7 (C═O), 141.3 (C⁶), 128.6, 128.4 (C^{2′}), 122.7, 122.6 (C^{3′}), 108.9 (C⁵), 81.7, 81.6 (O—CH₂—O), 45.1 (2C, C^{1′}), 38.8 (C(CH₃)₃), 27.0 and 25.6 (C^{4′}), 26.8 (C(CH₃)₃).

³¹P NMR (162 MHz, CDCl₃) δ 26.4

HRMS (ESI): m/z [M+Na]⁺ calcd for C20H30N2NaO9ClP: 531.1275, found: 531.1276.

### 1.9. (E)-N¹-(4′-bis(POM)-phosphinyl-2′-butenyl)-5-bromo-uracil (Ie1)

IR: 2976, 2361, 1751, 1693, 1441, 1235, 1137, 965, 854, 768 cm⁻¹.

¹H NMR (400 MHz, CD₃OD) δ 7.98 (s, 1H, H⁶), 5.85-5.76 (m, 1H, H^{2′}), 5.75-5.62 (m, 5H, O—CH₂-0, H^{3′}), 4.36 (t, J=5.1 Hz, 2H, H″), 2.82 (dd, J=22.5, 6.9 Hz, 2H, H^{4′}), 1.23 (s, 18H, tBu).

¹³C NMR (100 MHz, CD₃OD) δ 178.1 (C═O), 162.1 (C═O), 152.0 (C═O), 146.2 (C⁶), 131.4, 131.3 (C^{2′}), 124.5, 124.4 (C^{3′}), 96.8 (C⁵), 83.3, 83.2 (O—CH₂—O), 50.6, 50.5 (C^{1′}), 39.8 (C(CH₃)₃), 31.9, 30.5 (C^{4′}), 27.2 (C(CH₃)₃).

³¹P NMR (162 MHz, CD₃OD) δ 27.1.

HRMS (ESI): m/z [M+Na]⁺ calcd for C₂₀H30N2NaO9BrP: 575.0770, found: 575.0764.

### 1.10. (Z)—N¹-(4′-bis(POM)-phosphinyl-2′-butenyl)-5-bromo-uracil (Ie2)

¹H NMR (400 MHz, CD₃OD) δ 8.06 (s, 1H, H⁶), 5.70-5.52 (m, 6H, O—CH₂—O, H^{2′}, H^{Y}), 4.45 (dd, J=6.8, 3.7 Hz, 2H, H″), 3.01 (dd, J=23.4, 7.8 Hz, 2H, H^{4′}), 1.24 (s, 18H, tBu).

¹³C NMR (100 MHz, CD₃OD) δ 178.2 (C═O), 162.2 (C═O), 152.1 (C═O), 146.3 (C⁶), 130.1, 129.9 (C^{2′}), 123.5, 123.4 (C^{3′}), 96.7 (C⁵), 83.2, 83.1 (O—CH₂—O), 46.2 (2C, C^{1′}), 39.8 (C(CH₃)₃), 27.7 and 26.3 (C^{4′}), 27.3 (C(CH₃)₃).

³¹P NMR (100 MHz, CD₃OD) δ 27.5.

HRMS (ESI): m/z [M+Na]⁺ calcd for C20H30N2NaO9BrP: 575.0770, found: 575.0778.

### 1.11. (E)-N-1-(4′-bis(POC)-phosphinylbut-2′-enyl)-5-(1,2-diiodovinyl)uracil (Iac1)

NMR ¹H (400 MHz, CDCl₃) δ 8.35 (s, 1H, H_{NH}), 7.38 (s, 1H, H₆), 7.33 (s, 1H, H_{CHI}), 5.79-5.61 (m, 6H, H_{O—CH2-O}, H_{2′} and H_{3′}), 4.95 (sept, J=6.3 Hz, 2H, H_{CHisopropyl}) 4.38 (t, J=4.6 Hz, 2H, H_{1′}), 2.78 (dd, J=23.1, 5.5 Hz, 2H, H_{4′}), 1.33 (d, J=6.3 Hz, 12H, H_{CH3isopropyl})

NMR ¹³C (100 MHz, CDCl₃) δ 159.1, 153.0, 149.7, 148.7, 140.4, 129.3, 129.2, 124.5, 124.4, 109.0, 116.58, 85.4, 84.2, 84.1, 73.4, 49.4, 31.3, 29.9, 21.6, 21.5

IR (cm⁻¹): 2985, 1756, 1679, 1467, 1257, 1152, 1101, 982, 950, 831, 788

NMR ³¹P (400 MHz, CDCl₃) δ 26.70

HRMS (M+Na): found 778.9355 calculated for C₂₀H₂₇N₂O₁₁PNa 778.9340

### 1.12. (E)-N-1-(4′-bis(POC)-phosphinylbut-2′-enyl)-5-(1-bromo-2-iodovinyl) uracil (Iac2)

NMR ¹H (400 MHz, CDCl₃) δ 8.26 (s, 1H, H_{NH}), 7.42 (s, 1H, H₆), 7.15 (s, 1H, H_{═CHI})_{,} 5.85-5.61 (m, 6H, H_{O—CH2-O}, H_{2′} and H_{3′}), 4.95 (sept, J=6.3 Hz, 2H, H_{CHisopropyl}), 4.42 (t, J=4.6 Hz, 2H, H_{1′}), 2.76 (dd, J=23.1, 5.5 Hz, 2H, H_{4′}), 1.36 (d, J=6.3 Hz, 12H, H_{CH3isopropyl})

NMR ¹³C (100 MHz, CDCl₃) δ 159.2, 153.2, 149.7, 148.7, 140.4, 129.3, 129.2, 124.5, 124.4, 109.0, 116.58, 85.4, 84.2, 84.1, 73.4, 49.4, 31.3, 29.9, 21.6, 21.5

NMR ³¹P (162 MHz, CDCl₃) δ 26.80

IR (cm⁻¹): 987, 1756, 1694, 1468, 1259, 1152, 1101, 981, 949, 870, 831, 787

HRMS (M+Na): found 732.2228 calculated for C₂₀H₂₇N₂O₁₁PNaIBr 732.2230

### 1.13. (E)-N-1-(4′-bis(POC)-phosphinylbut-2′-enyl)-5-(1-chloro-2-iodorovinyl) uracil (Iac3)

NMR ¹H (400 MHz, CDCl₃) δ 8.63 (s, 1H, H_{NH}), 7.45 (s, 1H, H₆), 6.91 (s, 1H, H_{═CHI}), 5.83-5.59 (m, 6H, H_{O—CH2-O}), H_{2′}, H_{3′}), 4.96 (sept, J=6.3 Hz, 2H, H_{CHisopropyl}), 4.41 (t, J=4.6 Hz, 2H, H_{1′}), 2.80 (dd, J=23.2, 5.5 Hz, 2H, H_{4′}), 1.35 (d, J=6.3 Hz, 12H, H_{CH3isopropyl}),

NMR ¹³C (100 MHz, CDCl₃) δ 159.3, 152.9, 149.7, 148.9, 140.4, 129.3, 129.0, 124.5, 124.3, 109.0, 116.58, 85.5, 84.7, 84.1, 73.6, 49.4, 31.3, 29.9, 21.4, 21.3

NMR ³¹P (162 MHz, CDCl₃) δ 26.70

IR (cm⁻¹): 2986, 1756, 1686, 1451, 1348, 1258, 1152, 1186, 981, 949, 903, 869, 831, 787

HRMS (M+H): found 665.0179 calculated for C₂₀H₂₈N₂O₁₁PClI 665.0164

### 1.14. (E)-N-1-(4′-bis(POC)-phosphinylbut-2′-enyl)-5-(1,2-dibromovinyl) uracil (Iac4)

NMR ¹H (400 MHz, CDCl_{3 δ} 8.54 (s, 1H, H_{NH}), 7.40 (s, 1H, H₆), 6.86 (s, 1H, H_{═CHBr}), 5.79-5.61 (m, 6H, H_{O—CH2-O}, H_{2′}, H₃₊), 4.93 (sept, J=6.3 Hz, 2H, H_{CHisopropyl}), 4.39 (t, J=4.6 Hz, 2H, H_{1′}), 2.77 (dd, J=23.0, 5.6 Hz, 2H, H_{4′}), 1.32 (d, J=6.3 Hz, 12H, H_{CH3isopropyl})

NMR ¹³C (100 MHz, CDCl₃) δ 159.1, 153.0, 149.7, 148.7, 140.4, 129.3, 129.2, 124.5, 124.4, 109.0, 116.58, 85.4, 84.2, 84.1, 73.4, 49.4, 31.3, 29.9, 21.6, 21.5

IR (cm⁻¹): 2986, 1756, 1691, 1442, 1347, 1260, 1153, 1186, 1029, 983, 951, 871, 832, 789

NMR ³¹P (162 MHz, CDCl_{3 δ} 26.80

HRMS (M+Na): found 685.7984 calculated for C₂₀H₂₇N₂O₁₁PNaBr₂ 685.7988

### EXAMPLE 2

### Synthesis of Purine Phosphonates Derivatives

The synthesis is illustrated in the following scheme

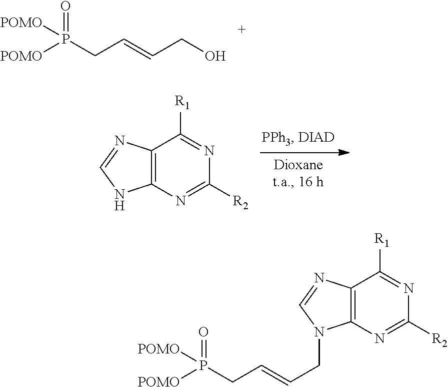

According to said procedure, the following compounds are obtained and characterised

### 2.1. N⁹-(4′-bis(POM)-phosphinyl-2′-butenyl)adenine (Iv)

To a dioxane solution (1.5 mL) of bis(POM)-1-hydroxymethyl-allylphosphonate (0.131 mmol), adenine (0.326 mmol), and triphenylphosphine (0.326 mmol) under argon was then added diisopropylazodicarboxylate (0.326 mmol). After 20 h stirring, volatiles were evaporated, and residue was purified by silica gel column chromatography to give desired compound (0.074 mmol, 57%).

¹H NMR (400 MHz, CDCl₃): δ 8.36 (s, 1H, H²), 7.81 (s, 1H, H⁸), 5.95-5.85 (m, 1H, H^{2′}), 5.74-5.60 (m, 7H, H^{3′}, O—CH₂—O, NH₂), 4.80 (t, J=5.0 Hz, 2H, H^{1′}), 2.72 (dd, J=22.6, 7.3 Hz, 2H, H^{4′}), 1.23 (s, 18H, C(CH₃)₃).

¹³C NMR (100 MHz, CDCl₃): δ 176.8 (C═O), 155.4 (C⁶), 153.1 (C²), 149.9 (C⁴), 140.1 (C⁸), 130.0, 129.8 (C^{2′}), 123.4, 123.2 (C^{3′}), 119.6 (C⁵), 81.6 (2C, O—CH₂—O), 44.9 (2C, C^{1′}), 38.7 (C(CH₃)₃), 31.5, 30.1 (C^{4′}), 26.8 (C(CH₃)₃). ³¹P NMR (162 MHz, CDCl₃): δ 26.50

HRMS (ESI): m/z [M+H]⁺ calcd for C₂₁H₃₃N₅O₇P: 498.2118, found: 498.2127.

### 2.2. N⁹-(4′-bis(POM)-phosphinyl-2′-butenyl)-6-chloropurine (Iw)

¹H NMR (400 MHz, CDCl₃): δ 8.73 (s, 1H, H²), 8.14 (s, 1H, H⁸), 5.94-5.85 (m, 1H, H^{2′}), 5.80-5.71 (m, 1H, H^{3′}), 5.71-5.67 (m, 4H, O—CH₂—O), 4.88 (t, J=5.2 Hz, 2H, H″), 2.71 (dd, J=22.8, 7.1 Hz, 2H, H^{4′}), 1.21 (s, 18H, C(CH₃)₃).

¹³C NMR (100 MHz, CDCl₃): δ 176.8 (C═O), 152.0 (C²), 151.6, 151.1 (C⁴ and C⁶), 144.7 (C⁸), 131.6 (C⁵), 128.9, 128.7 (C^{2′}), 124.6, 124.5 (C^{3′}), 81.6, 81.5 (O—CH₂—O), 45.5, 45.4 (C^{1′}), 38.7 (C(CH₃)₃), 31.4, 30.0 (C^{4′}), 26.8 (C(CH₃)₃). ³¹P NMR (162 MHz, CDCl₃): δ 26.05.

HRMS (ESI): m/z [M+H]⁺ calcd for C21H30N4O7PCl: 539.1438, found: 539.1449.

### 2.3. N⁹-(4′-bis(POM)-phosphinyl-2′-butenyl)-2-amino-6-chloropurine (Ix)

¹H NMR (400 MHz, CDCl₃): δ 7.74 (s, 1H, H⁸), 5.88-5.79 (m, 1H, H^{2′}), 5.72-5.59 (m, 5H, H^{3′}, O—CH₂—O), 5.28 (s, 2H, NH₂), 4.65 (t, J=5.1 Hz, 2H, H″), 2.70 (dd, J=22.7, 7.2 Hz, 2H, H^{4′}), 1.20 (s, 18H, C(CH₃)₃).

¹³C NMR (100 MHz, CDCl₃): δ 176.8 (C═O), 159.1 (C⁶), 153.6 (C⁴), 151.3 (C²), 141.8 (C⁸), 129.4, 129.3 (C^{2′}), 125.1 (C⁵), 123.6, 123.5 (C^{3′}), 81.6, 81.5 (O—CH₂—O), 44.9 (C^{1′}), 38.7 (C(CH₃)₃), 31.4, 30.0 (C^{4′}), 26.8 (C(CH₃)₃). ³¹P NMR (162 MHz, CDCl₃): δ 26.35.

HRMS (ESI): m/z [M+Na]⁺ calcd for C₂₁H₃₁N₅O₇NaPCl: 554.1547, found: 554.1566.

2.4. N⁹-(4′-bis(POM)-phosphinyl-2′-butenyl)-6-cyclopropylaminopurine (Iy)

A solution of compound Iw (0.046 mmol) in a mixture of cyclopropylamine (0.2 mL) and dichloromethane (2 mL) was stirred for 20 h. After evaporation of all volatiles, the residue was purified by silica gel column chromatography to give desired compound (0.038 mmol, 82%).

¹H NMR (400 MHz, CDCl₃): δ 8.47 (s, 1H, H²), 7.75 (s, 1H, H⁸), 5.95 (s, 1H, NH), 5.93-5.84 (m, 1H, H^{2′}), 5.69-5.61 (m, 5H, H^{3′}, O—CH₂—O), 4.78 (t, J=5.1 Hz, 2H, H″), 3.04 (d, J=3.0 Hz, 1H, NHCH), 2.70 (dd, J=22.6, 7.3 Hz, 2H, H′^{1′}), 1.21 (s, 18H, C(CH₃)₃), 0.94 (td, J=8.4, 6.9 Hz, 2H, NHCHCH₂), 0.68-0.64 (m, 2H, NHCHCH₂).

¹³C NMR (100 MHz, CDCl₃): δ 176.8 (C═O), 155.8 (C⁶), 153.3 (C²), 149.0 (C⁴), 139.5 (C⁸), 130.1, 129.9 (C^{2′}), 123.2, 123.0 (C^{3′}), 119.8 (C⁵), 81.6, 81.5 (O—CH₂—O), 44.8 (2C, C^{1′}), 38.7 (C(CH₃)₃), 31.5, 30.1 (C^{4′}), 26.8 (C(CH₃)₃), 23.7 (NHCH), 7.4 (NHCHCH₂).

³¹P NMR (162 MHz, CDCl₃): δ26.57.

HRMS (ESI): m/z [M+H]⁺ calcd for C₂₄H₃₇N₅O₇P: 538.2431, found: 538.2430.

### 2.5. N⁹-(4′-bis(POM)-phosphinyl-2′-butenyl)-2-amino-6-cyclopropylaminopurine (Iz)

A solution of compound Ix (0.037 mmol) in mixture of cyclopropylamine (0.2 mL) and dichloromethane (2 mL) was stirred for 20 h. After evaporation of all volatiles, the residue was purified by silica gel column chromatography to give desired compound (0.029 mmol, 77%).

¹H NMR (400 MHz, CDCl₃): δ 7.44 (s, 1H, H²), 5.89-5.81 (m, 1H, H^{2′}), 5.76 (s, 1H, NH), 5.67-5.57 (m, 5H, H^{3′}, O—CH₂—O), 4.79 (s, 2H, NH₂), 4.61 (t, J=5.1 Hz, 2H, H^{Y}), 3.05-2.95 (m, 1H, NHCH), 2.69 (dd, J=23.0, 7.0 Hz, 2H, H^{4′}), 1.22 (s, 18H, C(CH₃)₃), 0.85 (td, J=6.9, 5.4 Hz, 2H, NHCHCH₂), 0.63-0.58 (m, 2H, NHCHCH₂).

¹³C NMR (100 MHz, CDCl₃): δ 176.8 (C═O), 160.1, 156.3 (C⁶ and C²), 151.0 (C⁴), 136.9 (C⁸), 130.6, 130.4 (C^{2′}), 122.4, 122.3 (C^{3′}), 114.6 (C⁵), 81.6 (2C, O—CH₂—O), 44.4, 44.3 (2C, C″), 38.7 (C(CH₃)₃), 31.4, 30.0 (C^{4′}), 26.8 (C(CH₃)₃), 23.7 (NHCH), 7.4 (NHCHCH₂). ³¹P NMR (162 MHz, CDCl₃): δ 26.77.

HRMS (ESI): m/z [M+H]⁺ calcd for C₂₄H₃₈N₆O₇P: 553.2531, found: 553.2540.

2.6. N⁹-(4′-bis(POM)-phosphinyl-2′-butenyphypoxanthine (Iaa)

A solution of compound by (0.046 mmol) in a mixture of water (0.75 mL) and formic acid (0.75 mL) was stirred for 20 h. After evaporation of all volatiles, the residue was purified by silica gel column chromatography to give desired compound (0.040 mmol, 86%).

¹H NMR (400 MHz, CDCl₃): δ 12.94 (s, 1H, NH), 8.17 (s, 1H, H²), 7.81 (s, 1H, H⁸), 5.94-5.83 (m, 1H, H^{2′}), 5.78-5.60 (m, 5H, H^{3′}, O—CH₂—O), 4.78 (t, J=5.2 Hz, 2H, H″), 2.72 (dd, J=22.7, 7.2 Hz, 2H, H^{4′}), 1.21 (s, 18H, C(CH₃)₃).

¹³C NMR (100 MHz, CDCl₃): δ 176.8 (C═O), 159.1 (C⁶), 148.9 (C⁴), 145.0 (C²), 139.7 (C⁸), 129.7, 129.5 (C^{2′}), 124.5 (C⁵), 123.8, 123.7 (C^{3′}), 81.6 (2C, O—CH₂—O), 45.3 (2C, C^{Y}), 38.7 (C(CH₃)₃), 31.4, 30.0 (C^{4′}), 26.8 (C(CH₃)₃). ³¹P NMR (162 MHz, CDCl₃): δ 26.40.

HRMS (ESI): m/z [M+Na]⁺ calcd for C24H26N4O8Na: 521.1648, found: 521.1625.

### 2.7. N⁹-(4′-bis(POM)-phosphinyl-2′-butenyl)guanine (Iab)

A solution of compound Ix (0.041 mmol) in a (1:1) mixture of water (0.75 mL) and formic acid (0.75 mL) was stirred for 20 h. After evaporation of all volatiles, the residue was purified by silica gel column chromatography to give desired compound (0.035 mmol, 86%).

¹H NMR (400 MHz, CDCl₃): δ 12.11 (s, 1H, NH), 7.60 (s, 1H, H⁸), 6.65 (s, 2H, NH₂), 5.95-5.84 (m, 1H, H^{1′}), 5.73-5.62 (m, 5H, H^{3′}, O—CH₂—O), 4.60 (t, J=4.7 Hz, 2H, H″), 2.72 (dd, J=22.6, 7.3 Hz, 2H), 1.20 (s, 18H, C(CH₃)₃).

¹³C NMR (100 MHz, CDCl₃): δ 176.9 (C═O), 159.0 (C⁶), 153.9 (C²), 151.4 (C⁴), 137.2 (C⁸), 130.6, 130.4 (C^{2′}), 122.7, 122.6 (C^{3′}), 116.8 (C⁵), 81.7, 81.6 (O—CH₂—O), 44.8 (C^{1′}), 38.7 (C(CH₃)₃), 31.4, 30.0 (C^{4′}), 26.8 (C(CH₃)₃).

³¹P NMR (100 MHz, CDCl₃): δ 27.04.

HRMS (ESI): m/z [M+Na]⁺ calcd for C21H32N5O8NaP: 536.1886, found: 536.1890.

### 2.8. N9-(4′-bis(POM)-phosphinylbut-2′-enyl)-6-allylaminopurine (Iad)

From 6-cyclopropylaminopurine by using general procedure, compound (Ia2) was obtained as a colourless oil (52%).

NMR ¹H (400 MHz, CDCl₃) δ 8.39 (s, 1H, H₂), 7.75 (s, 1H, H₈), 6.10-5.76 (m, 1H, H_{2′}), 5.75-5.54 (m, 5H, H_{3′}, H_{O—CH2-O}), 5.25 (ddd, J=13.7, 11.6, 1.4 Hz, 2H, H_{C3}), 4.78 (t, J=5.0 Hz, 2H, H_{1′}), 4.33 (d, J=11.6 Hz, 2H, H_{C1}), 2.71 (dd, J=22.6, 7.2 Hz, 2H, H_{4′}), 1.21 (s, 18H, H_{C(CH3)3})

NMR ¹³C (100 MHz, CDCl₃) δ 176.26, 175.83, 158.08, 152.42, 151.06, 141.07, 136.02, 133.44, 126.22, 121.87, 114.80, 83.85, 82.95, 46.24, 41.99, 39.86, 39.47, 32.31, 26.99, 26.78

NMR ³¹P (162 MHz, CDCl₃) δ 26.57

HRMS (M⁺H) found 538.2440 calculated for C₂₄H₃₇N₅O₇P: 538.2431

### 2.9. N-9-(4′-bis(POM)-phosphinylbut-2′-enyl)-2-amino-6-methoxypurine (Iae)

From 2-amino-6-methyoxypurine, by using general procedure compound (Iae) was obtained as a colourless oil (51%).

NMR ¹H (400 MHz, CDCl₃) δ 8.04 (s, 1H, H₈), 5.73-5.61 (m, 6H, H_{2′}, H_{3′}, H_{O—CH2-O}), 4.76 (d, J=5.3 Hz, 1H, H_{1′a}), 4.56 (d, J=5.4 Hz, 1H, H_{1′b}), 3.99 (s, 3H, H_{OMe}), 3.02 (s, 2H, H_{NH2}), 2.66 (dd, J=11.9, 5.4 Hz, 2H, H_{4′}), 1.26 (s, 18H, H_{C(CH3)3})

NMR ¹³C (100 MHz, CDCl₃) δ 176.04, 164.04, 160.79, 149.49, 137.51, 133.43, 127.28, 121.87, 83.40, 53.95, 46.23, 39.66, 32.65, 31.97, 26.88

NMR ³¹P (162 MHz, CDCl₃) δ 26.51

HRMS (M⁺H) found 528.2228 calculated for C₂₂H₃₅N₅O₈P: 528.2223

### 2.10 N-9-(4′-bis(POM)-phosphinylbut-2′-enyl)-6-(4-isopropyl)phenyl aminopurine (Iaf)

From 6-(4-isopropyl)phenylaminopurine, by using general procedure, compound (Iaf) was obtained as a colourless oil (39%).

NMR ¹H (400 MHz, CDCl₃) δ 8.67 (s, 1H, H₂), 8.19 (s, 1H, H₈), 7.22 (d, J=7.5 Hz, 2H, Hₐᵣₒₘ), 6.93 (d, J=7.5 Hz, 2H, Hₐᵣₒₘ), 5.72-5.59 (m, 6H, H_{2′}, H_{3′}, H_{O—CH2-O}), 4.96 (d, J=5.4 Hz, 1H, H_{1′a}), 4.48 (d, J=5.2 Hz, 1H, H_{1′b}), 3.86 (s, 1H, H_{NH}), 3.04 (hept, J=6.4 Hz, 1H, H_{CHisopropyl}), 2.66 (dd, J=11.9, 5.4 Hz, 2H, H_{4′}), 1.34 (d, J=6.4 Hz, 6H, H_{CH3isopropyl}), 1.30 (s, 18H, H_{C(CH3)3})

NMR ¹³C (100 MHz, CDCl₃) δ 176.04, 153.12, 151.64, 150.82, 143.37, 141.06, 138.38, 133.43, 127.76, 123.52, 123.04, 121.87, 83.40, 46.23, 39.66, 34.19, 32.65, 31.97, 26.88, 23.37

NMR ³¹P (162 MHz, CDCl₃) δ 26.48

HRMS (M⁺H) found 616.2909 calculated for C₃₀H₄₃N₅O₇P: 616.2900

### 2.11. N-9-(4′-bis(POM)-phosphinylbut-2′-enyl)-6-cyclohexylaminopurine (Iag)

From 6-cyclohexylaminopurine, by using general procedure, compound (Iag) was obtained as a colourless oil (42%).

NMR ¹H (400 MHz, CDCl₃) δ 8.39 (s, 1H, H₂), 8.14 (s, 1H, H₈), 5.73-5.59 (m, 6H, H_{2′}, H_{3′}, H_{O—CH2-O}), 4.97 (d, J=5.4 Hz, 1H, H_{1′a}), 4.48 (d, J=5.4 Hz, 1H, H_{1′b}), 3.78 (ft, J=7.9, 3.8 Hz, 1H, H_{CHcyclohexyl}), 2.66 (dd, J=11.9, 5.4 Hz, 2H, H_{4′}), 1.92 (dqd, J=11.4, 5.7, 2.1 Hz, 2H, H_{CH2cyclohexyl}), 1.78 (s, 1H, H_{NH}), 1.72 (tdd, J=11.5, 5.7, 2.2 Hz, 2H, H_{CH2cyclohexyl})_{,} 1.67-1.52 (m, 6H, H_{CH2cyclohexyl}), 1.28 (s, 18H, H_{C(CH3)3})

NMR ¹³C (100 MHz, CDCl₃) δ 176.04, 157.04, 151.05, 150.21, 141.06, 133.43, 124.13, 121.87, 83.40, 52.78, 46.23, 39.66, 33.31, 32.65, 31.97, 26.88, 25.91, 24.72

NMR ³¹P (162 MHz, CDCl₃) δ 26.54

HRMS (M⁺H) found 580.6410 calculated for C₂₇H₄₃N₅O₇P: 580.6417

### 2.12. N-9-(4′-bis(POM)-phosphinylbut-2′-enyl)-6-butylaminopurine (Iah)

From 6-n-butylaminopurine, by using general procedure, compound (Iah) was obtained as a colourless oil (49%).

NMR ¹H (400 MHz, CDCl₃) δ 8.32 (s, 1H, H₂), 8.02 (s, 1H, H₈), 5.71-5.58 (m, 6H, H_{2′}, H_{3′}, H_{O—CH2-O),} 4.85 (d, J=5.3 Hz, 1H, H_{1′a}), 4.74 (d, J=5.4 Hz, 1H, H_{1′b}), 3.44 (t, J=4.9 Hz, 1H, H_{C1a}), 3.35 (t, J=4.9 Hz, 1H, H_{C1b}), 2.66 (dd, J=11.9, 5.4 Hz, 2H, H_{4′}), 2.01 (s, 1H, H_{NH}), 1.58 (ddt, J=12.8, 7.6, 5.0 Hz, 2H, H_{C2}), 1.46-1.36 (m, 2H, H_{C3}), 1.28 (s, 18H, H_{C(CH3)3}), 0.99 (t, J=6.6 Hz, 3H, H_{C4})

NMR ¹³C (100 MHz, CDCl₃) δ 176.04, 157.21, 152.47, 151.00, 141.06, 133.43, 125.05, 121.87, 83.40, 46.23, 43.71, 39.66, 32.65, 31.97, 30.87, 26.88, 20.22, 14.01

NMR ³¹P (162 MHz, CDCl₃) δ 26.48

HRMS (M³⁰ H) found 554.2739 calculated for C₂₅H₄₁N₅O₇P: 554.2744

### 2.13. N-9-(4′-bis(POM)-phosphinylbut-2′-enyl)-6-phenylthiopurine (Iai)

From 6-phénylthiopurine, by using general procedure, compound (Iai) was obtained as a colourless oil (38%).

NMR ¹H (400 MHz, CDCl₃) δ 8.60 (s, 1H, H₂), 7.96 (s, 1H, H₈), 7.18 (dd, J=7.5, 1.5 Hz, 2H, Hₐᵣₒₘ), 7.12 (t, J=7.4 Hz, 2H, Hₐᵣₒₘ), 7.06-7.00 (m, 1H, Hₐᵣₒₘ), 5.75-5.62 (m, 6H, H_{2′} H_{3′}, H_{O—CH2-O}), 4.74 (d, J=5.3 Hz, 1H, H_{1′}), 4.53 (d, J=5.3 Hz, 1H, H₈), 2.66 (dd, J=11.9, 5.4 Hz, 2H, H_{4′}), 1.29 (s, 18H, H_{C(CH3)3})

NMR ¹³C (100 MHz, CDCl₃) δ 176.04, 166.24, 150.60, 144.49, 142.89, 136.74, 135.37, 133.43, 131.46, 129.78, 129.43, 121.87, 83.40, 46.23, 39.66, 32.65, 31.97, 26.88

NMR ³¹P (162 MHz, CDCl₃) δ 26.49

HRMS (M⁺H) found 591.6321 calculated for C₂₇H₃₆N₄O₇PS: 591.6329

### 2.14. N-9-(4′-bis(POM)-phosphinylbut-2′-enyl)-2,6-diaminopurine (Iaj)

From 2,6-diaminopurine, by using general procedure compound (Iaj) was obtained as a colourless oil (41%).

NMR ¹H (400 MHz, CDCl₃) δ 7.50 (s, 1H, H₈), 5.94-5.76 (m, 1H, H_{2′}), 5.71-5.58 (m, 5H, H_{3′} and H_{O—CH2-O}), 5.54 (s, 2H, H_{NH2}), 4.80 (s, 2H, H_{NH2}), 4.61 (t, J=5.2 Hz, 2H, H_{1′}), 2.69 (dd, J=22.6, 7.2 Hz, 2H, H_{4′),} 1.21 (s, 18H, H_{C(CH3)3})

NMR ¹³C (100 MHz, CDCl₃) δ 176.26, 175.83, 164.27, 159.42, 149.75, 140.52, 133.44, 122.34, 121.87, 83.85, 82.95, 46.24, 39.86, 39.47, 32.31, 26.99, 26.78

NMR ³¹P (162 MHz, CDCl₃) δ 26.42

HRMS (M⁺H) found 513.2225 calculated for C₂₁H₃₄N₆O₇P: 513.2227

### 2.15. N-9-(4′-bis(POM)-phosphinylbut-2′-enyl)-2-amino-6-allylaminopurine (Iak)

From 2-amino-6-allylaminopurine, by using general procedure compound (Iak) was obtained as a colourless oil (40%).

NMR ¹H (400 MHz, CDCl₃) δ 8.08 (s, 1H, H₈), 5.79 (ddt, J=16.4, 10.1, 6.2 Hz, 1H, H_{C2}), 5.72-5.60 (m, 6H, H_{2′, H}_{3′}, H_{O—CH2-O}), 5.12 (ddd, J=8.8, 4.7, 1.0 Hz, 2H, H_{C3}), 4.98 (d, J=5.5 Hz, 1H, H_{1′a}), 4.48 (d, J=5.4 Hz, 1H, H_{1′b}), 4.22 (d, J=6.2 Hz, 1H, H_{C1a}), 4.12 (d, J=6.0 Hz, 1H, H_{C1b}), 2.66 (dd, J=11.9, 5.5 Hz, 2H, H_{4′}), 2.12 (s, 1H, H_{NH}), 1.29 (s, 18H, H_{C(CCH3)3})

NMR ¹³C (100 MHz, CDCl₃) δ 176.26, 175.83, 161.42, 160.16, 150.97, 141.07, 136.02, 133.44, 123.91, 121.87, 114.80, 83.85, 82.95, 46.24, 41.99, 39.86, 39.47, 32.31, 26.99, 26.78

NMR ³¹P (162 MHz, CDCl₃) δ 26.52

HRMS (M⁺H) found 553.4687 calculated for C₂₄H₃₈N₆O₇P: 553.4688

### 2.16. N-9-(4′-bis(POM)-phosphinylbut-2′-enyl)-2-amino-6-(4-isopropyl)phenylamino purine (Ial)

From 2-amino-6-(4-isopropyl)phenylaminopurine, by using general procedure, compound (Ial) was obtained as a colourless oil (45%).

NMR ¹H (400 MHz, CDCl₃) δ 7.88 (s, 1H, H₂), 7.22 (d, J=7.5 Hz, 2H, Hₐᵣₒₘ), 6.93 (d, J=7.5 Hz, 2H, Hₐᵣₒₘ), 5.71-5.58 (m, 6H, H_{2′}, H_{3′}, H_{O—CH2-O}), 4.91 (d, J=5.2 Hz, 1H, H_{1′a}), 4.49 (d, J=5.4 Hz, 1H, H_{1′b}), 3.89 (s, 1H, H_{NH}), 3.04 (hept, J=6.3 Hz, 1H, H_{CHisopropyl}), 2.66 (dd, J=11.9, 5.4 Hz, 2H, H_{4′}), 2.11 (s, 2H, H_{NH2}), 1.33 (d, J=6.4 Hz, 6H, H_{CH3isopropyl}), 1.30 (s, 18H, H_{C(CH3)3})

NMR ¹³C (100 MHz, CDCl₃) δ 176.04, 161.65, 155.21, 150.38, 143.37, 141.06, 138.38, 133.43, 127.76, 123.52, 122.63, 121.87, 83.40, 46.23, 39.66, 34.19, 32.65, 31.97, 26.88, 23.37

NMR ³¹P (162 MHz, CDCl₃) δ 26.42

HRMS (M⁺H) found 631.6754, calculated for C₃₀H₄₄N₆O₇P: 631.6748

### 2.17. N-9-(4′-bis(POM)-phosphinylbut-2′-enyl)-2-amino-6-cyclohexylaminopurine (Iam)

From 2-amino-6-cyclohexylaminopurine by using general procedure, compound (Iam) was obtained as a colourless oil (50%).

NMR ¹H (400 MHz, CDCl₃) δ 7.81 (s, 1H, H₈), 5.73-5.60 (m, 6H, H_{2′}, H_{3′}, H_{O—CH2-O}), 4.92 (d, J=5.3 Hz, 1H, H_{1′a}), 4.51 (d, J=5.3 Hz, 1H, H_{1′b}), 3.71-3.62 (m, 1H, H_{CH2cyclohexyl}), 2.66 (dd, J=11.9, 5.4 Hz, 2H, H_{4′}), 2.14-2.03 (m, 3H, H_{NH} and H_{CH2cyclohexyl}), 1.75-1.56 (m, 8H, H_{CH2cyclohexyl}), 1.30 (s, 18H, H_{C(CH3)3})

NMR ¹³C (100 MHz, CDCl₃) δ 176.04, 159.80, 159.56, 151.27, 141.06, 133.43, 122.02, 121.69, 83.40, 52.78, 46.23, 39.66, 33.31, 32.65, 31.97, 26.88, 25.91, 24.72

NMR ³¹P (162 MHz, CDCl₃) δ 26.51

HRMS (M⁺H) found 595.6322 calculated for C₂₇H₄₄N₆O₇P: 595.6333

### EXAMPLE 3

### Synthesis of Furano-Uracil Phosphonates Derivatives; General Procedure

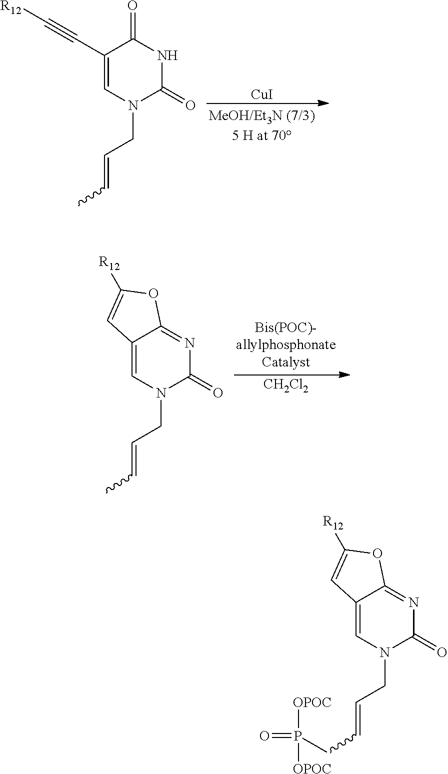

Linear compound is solubilized in methanol and triethylamine mixture (7/3). The solution is heated at 70° C. for 5 hours. After completion of the reaction (checked by TLC), solvent are removed under reduced pressure and the obtained crude product is purified on silica gel (eluent: petroleum ether ethyl acetate 4/6). To a CH₂Cl₂ (25 mL/mmol) solution of N¹-crotyl-4,5-substituted furano-uracil (1 equiv.), bis(POC) allylphosphonate (1.3 equiv.) and catalyst (0.05 eq) was added. This solution was stirred under positive pressure of dry argon. After evaporation of all volatiles, the residue was purified by silica gel column chromatography (EtOAc/Petroleum ether).

According to said procedures the following compounds are obtained and characterized

### 3.1. N-1-(4′-bis(POC)-phosphinylbut-2′-enyl)-4,5-((4-propylphenyl)furano)-uracil (Ian)

NMR ¹H (400 MHz, CDCl₃) δ 7.97 (s, 1H, H₆), 7.69 (d, J=8.2 Hz, 2H, Hₐᵣₒₘ), 7.35-7.18 (m, 2H, Hₐᵣₒₘ), 6.71 (s, 1H, H_{furano})_{,} 5.98-5.58 (m, 6H, H_{O—CH2-O}, H_{2′}, H_{3′}), 4.95 (sept, J=6.3 Hz, 2H, H_{CHisopropyl}), 4.38 (t, J=4.6 Hz, 2H, H_{1′}), 2.81 (dd, J=22.7, 6.6 Hz, 2H, H_{4′}), 2.64 (t, J=7.6 Hz, 2H, H_{C1}), 1.68 (dq, J=14.6, 7.3 Hz, 2H, H_{C2}), 1.32 (d, J=6.3 Hz, 12H, H_{CH3isopropyl}), 0.97 (t, J=7.3 Hz, 3H, H_{C3})

NMR ¹³C (100 MHz, CDCl₃) δ 171.44, 162.33, 155.38, 154.95, 151.54, 144.56, 143.62, 131.59, 130.92, 127.57, 127.18, 124.39, 124.18, 122.27, 102.19, 85.18, 84.27, 72.53, 72.22, 51.10, 38.12, 32.31, 24.48, 22.65, 22.44, 12.98

NMR ³¹P (162 MHz, CDCl₃) δ 27.00

HRMS (M+Na): found 643.2049 calculated for C₂₉H₃₇N₂O₁₁NaP 643.2033

### 3.2. N-1-(4′-bis(POC)-phosphinylbut-2′-enyl)-4,5-((4-pentylphenyl)furano)-uracil (Iao)

NMR ¹H (400 MHz, CDCl₃) δ 7.96 (s, 1H, H₆), 7.69 (d, J=8.2 Hz, 2H, Hₐᵣₒₘ), 7.27 (d, J=8.1 Hz, 2H, Hₐᵣₒₘ), 6.70 (s, 1H, H_{furano}), 5.97-5.61 (m, 6H, H_{O—CH2-O}, H_{2′}, H_{3′}), 4.93 (kept, J=6.3 Hz, 2H, H_{CHisopropyl}), 4.72-4.62 (t, J=4.6 Hz, H_{1′}), 2.81 (dd, J=22.7, 6.6 Hz, 2H, H_{4′}), 2.71-2.59 (m, 2H, H_{C1}), 1.65 (dt, J=15.1, 7.5 Hz, 2H, H_{C2}), 1.41-1.24 (m, 16H, H_{C3}, H_{C4}, H_{CH3isopropyl}), 0.92 (t, J=6.7 Hz, 3H, H_{C5})

NMR ¹³C (100 MHz, CDCl₃) δ 171.44, 162.33, 155.38, 154.95, 151.54, 143.94, 143.62, 131.59, 131.04, 128.08, 127.87, 124.31, 124.10, 122.27, 102.19, 85.18, 84.27, 72.53, 72.22, 51.10, 36.41, 32.31, 30.64, 30.02, 22.94, 22.65, 22.44, 14.02

NMR ³¹P (162 MHz, CDCl₃) δ 27.04

HRMS (M+Na): found 671.6524 calculated for C₃₁H₄₁N₂O₁₁NaP 671.6533

### 3.3. N-1-(4′-bis(POC)-phosphinylbut-2′-enyl)-4,5-(phenylethylfurano)-uracil (Iap)

NMR ¹H (400 MHz, CDCl₃) δ 7.81 (s, 1H, H₆), 7.37-7.15 (m, 5H, Hₐᵣₘₒ), 6.09 (s, 1H, H_{furano}), 5.95-5.58 (m, 6H, H_{O—CH2-O}, H_{2′}, H_{3′}), 4.90 (hept, J=6.2 Hz, 2H, H_{CHisopropyl}), 4.67 (t, J=4.7 Hz, H_{1′}), 3.10-2.93 (m, 4H, H_{C1}, H_{C2}), 2.80 (dd, J=22.7, 6.7 Hz, 2H, H_{4′}), 1.34 (d, J=6.3 Hz, 12H, H_{CH3isopropyl})

NMR ¹³C (100 MHz, CDCl₃) δ 163.72, 162.33, 155.38, 154.95, 148.01, 141.00, 138.22, 131.59, 129.05, 128.65, 126.53, 122.27, 108.20, 103.63, 85.18, 84.27, 72.53, 72.22, 51.10, 33.25, 28.37, 22.65, 22.44

NMR ³¹P (162 MHz, CDCl₃) δ 27.10

HRMS (M+Na): found 643.2049 calculated for C₂₉H₃₇H₂O₁₁NaP 643.2033

### 3.4. N-1-(4′-bis(POC)-phosphinylbut-2′-enyl)-4,5-(octylfurano)-uracil (Iaq)

NMR ¹H (400 MHz, CDCl₃) δ 7.79 (s, 1H, H₆), 6.09 (s, 1H, H_{furano}), 5.88-5.57 (m, 6H, H_{O—CH2-O}, H_{2′}, H_{3′}), 4.89 (hept, J=6.3 Hz, 2H, H_{CHisopropyl})_{,} 4.74 (t, J=4.5 Hz, H_{1′}), 2.75 (dd, J=22.7, 7.2 Hz, 2H, H_{4′}), 2.61 (t, J=7.4 Hz, 2H, H_{C1}), 1.70-1.59 (m, 2H, H_{C2}), 1.27 (m, 22H, H_{C3}, H_{C4}, H_{C5}, H_{C6}, H_{C7}, H_{CH3isopropyl}), 0.85 (t, J=6.8 Hz, 3H, H_{C8})

NMR ¹³C (100 MHz, CDCl₃) δ 171.95, 160.08, 155.36, 153.09, 137.92, 130.28, 123.88, 108.21, 98.54, 84.11, 73.43, 52.01, 31.77, 31.44, 30.05, 29.28, 28.82, 28.26, 26.73, 22.59, 21.60, 14.05

NMR ³¹P (162 MHz, CDCl₃) δ 27.07

HRMS (M+H): found 615.2682 calculated for C₂₈H₄₄N₂O₁₁P 615.2683

### 3.5. N-1-(4′-bis(POC)-phosphinylbut-2′-enyl)-4,5-(pentylfurano)-uracil (Iar)

NMR ¹H (400 MHz, CDCl₃) δ 7.79 (s, 1H, H₆), 6.09 (s, 1H, H_{furano}), 5.88-5.56 (m, 6H, H_{O—CH2-O}+H_{2′}+H_{3′}), 4.89 (hept, J=6.3 Hz, 2H, H_{CHisopropyl}), 4.63-4.55 (m, 2H, H_{1′}), 2.74 (dd, J=22.7, 7.1 Hz, 2H, H_{4′}), 2.61 (t, J=7.4 Hz, 2H, H_{C1}), 1.71-1.60 (m, 2H, H_{C2}), 1.37-1.24 (m, 16H, H_{C3}+H_{C4}+H_{CH3isopropyl}), 0.87 (dd, J=8.5, 5.6 Hz, 3H, H_{C5})

NMR ¹³C (100 MHz, CDCl₃) δ 171.94, 160.04, 155.36, 153.09, 137.97, 130.27, 123.87, 108.21, 98.57, 84.11, 73.42, 52.00, 31.44, 31.13, 30.05, 28.21, 26.40, 22.26, 21.59, 13.88

NMR ³¹P (162 MHz, CDCl₃) δ 27.06

HRMS (M+H): found 573.2208 calculated for C₂₅H₃₈N₂O₁₁P 573.2213

### 3.6. (E)-N¹-(4′-bis(POM)-phosphinyl-2′-methyl-but-2′-enyl) uracil (Iat)

To a CH₂Cl₂ (15 mL/mmol) solution of N¹-2-methyl-allyl uracil (0.60 mmol., 99.7 mg, 1.5 equiv.) and bis(POM) allylphosphonate (0.40 mmol., 140.1 mg, 1.0 equiv.) was added RuCl₂(PCy₃)IMesBenzylidene catalyst (0.020 mmol., 17 mg, 0.05 equiv.) then this solution was stirred at 40° C. for 16 h under positive pressure of dry air. After evaporation of all volatiles, the residue was purified by silica gel column chromatography to give the desired product

¹H NMR (400 MHz, MeOD) δ 7.46 (d, J=7.9, 1H), 5.69-5.61 (m, 5H), 5.34 (q, J=7.8, 1H), 4.33 (d, J=4.8, 2H), 2.82 (dd, J=22.9, 7.8, 2H), 1.69 (d, J=4.8, 3H), 1.23 (s, 18H).

¹³C NMR (101 MHz, MeOD) δ 178.53, 146.64, 102.89, 83.28, 55.04, 39.90, 27.39.

³¹P NMR (162 MHz, MeOD) δ 27.65.

HRMS (ESI): m/z [M+Na]⁺ calcd for C21H33N2NaO9P: 511.18159, found: 511.18159

### 3.7. (E)-N¹-(4′-bis(POM)-phosphinyl-but-2′-enyl)-cytosine (Ias)

To a DMF (1 mL) solution of (E)-4-bromomethyl-bis(POM)-allylphosphonate (0.25 mmol., 180 mg, 1.25 equiv.) was added to a suspension of sodium hydride (0.22 mmol., 5.3 mg, 1.1 equiv.) of cytosine (0.20 mmol., 18.5 mg, 1.0 equiv.) in DMF (1 mL) then this solution was stirred at 50° C. for 24 h under dry air. After evaporation of all volatiles, the residue was purified by silica gel column chromatography to give the desired products

¹H NMR (400 MHz, Acetone) δ 7.34 (d, J=6.8, 1H), 5.87 (d, J=6.8, 1H), 5.86-5.54 (m, 8H), 4.67 (t, J=4.9, 2H), 2.74 (dd, J=24.7, 9.3, 2H), 1.22 (s, 18H).

³¹P NMR (162 MHz, MeOD) δ 26.82.

HRMS (ESI): m/z [M+Na]⁺ calcd for C20H32N3NaO8P: 496.18192, found: 496.18214

### 3.8. (E)-N³-(4′-bis(POM)-phosphinyl-2′-butenyl)-3,9-dihydro-9-oxo-5H-imidazo[1,2-a]purine (Iau)

To a 1,4-dioxane/H₂O (2 mL, 1/1, v/v) solution of N9-(4′-bis(POM)-phosphinyl-2′-butenyl)-2-amino-6-chloropurine (116 mg, 0.218 mmol) was added 2-chloroacetaldehyde (2.5 mL, 50 wt. % in H2O). The resulting mixture was stirred 6 h at 70° C. and was extracted with ethyl acetate. Combined organic layers were dried over MgSO₄ and concentrated in vacuo. Chromatography over silica gel in CH₂Cl₂/MeOH 98:2 afforded 56 mg (48%) of desired product as a white solid.

¹H NMR (400 MHz, CDCl₃) δ 11.63 (s, 1H, NH), 7.66 (d, J=2.8 Hz, 1H, H₇), 7.64 (s, 1H, H₂), 7.25 (d, J=7.9 Hz, 1H, H₇), 5.85 (dq, J=16.0, 5.6 Hz, 1H, H_{2′}), 5.70-5.60 (m, 5H, H_{O—CH2-O}, H_{3′}), 4.65 (t, J=4.8 Hz, 2H, H_{1′}), 2.72 (dd, J=22.4, 7.2 Hz, 2H, H_{4′}), 1.18 (s, 18H).

NMR ¹³C (100 MHz, CDCl₃) δ 176.92, 152.28, 150.32, 146.12, 138.07, 130.32 (d, J=15 Hz), 122.52 (d, J=11.6 Hz), 115.91 (d, J=18.2 Hz), 107.35, 81.67 (d, J=6.2 Hz), 38.66, (d, J=2.1 Hz), 31.28, 29.89, 26.76.

³¹P NMR (162 MHz, CDCl₃) δ 26.81

HRMS (ESI): m/z [M+Na]⁺ calcd for C23H33N5O8P: 538.20613, found: 538.20578

### 3.9. (E)-N¹-(4′-bis(POM)-phosphinyl-but-2′-enyl)-4-propyl-[1,2,3]-triazol (Iav)

To a solution of alkyne (0.11 mmol., 14.4 mg, 1.3 equiv.) and (E,Z)-4-azido-bis(POM)-allylphosphonate (0.08 mmol., 34.1 mg) in H₂O/t-BuOH (1:1, 100 μL) were added Cu powder (0.40 mmol., 11.6 mg, 5.0 equiv.) and CuSO₄ (0.02 mmol., 5.0 mg, 0.25 equiv.). The resulting suspension was stirred 8 h at room temperature, then the mixture was diluted with EtOAc (1 mL), and purified by preparative thin layer chromatography to give (E)-Triazol-1-yl-bis(POM)-allylphosphonate (26 mg, 59%)

¹H NMR (400 MHz, CDCl₃) δ 7.75 (s, 1H, H_{triazol}), 7.73 (d, J=8.0 Hz, 2H, Hₐᵣₒₘ), 7.22 (d, J=8.0 Hz, 2H, Hₐᵣₒₘ), 5.95-5.87 (m, 1H, CH═), 5.82-5.70 (m, 1H, CH═), 5.69-5.63 (m, 4H, H_{O—CH2-O}, H_{3′}), 4.99 (t, J=5.2, 2H, H_{1′}), 2.74 (dd, J=22.8, 7.2, 2H, H_{4′}), 2.60 (t, J=7.6, 2H, CH₂), 1.65 (t, J=7.6, 3H, CH₃), 1.21 (s, 18H), 0.94 (t, J=7.6, 3H, CH₃).

NMR ¹³C (100 MHz, CDCl₃) δ 176.84, 148.22, 142.78, 129.20 (d, J=15.1 Hz), 128.87 127.94, 126.62, 124.46 (d, J=11.6 Hz), 118.95, 88.57 (d, J=6.2 Hz), 51.75 (d, J=2.1 Hz), 38.71, 37.79, 31.51, 30.11, 24.43, 13.75.

³¹P NMR (162 MHz, CDCl₃) δ 26.35

HRMS (ESI): m/z [M+Na]⁺ calcd for C27H41N3O7P: 550.26766, found: 550.26727

### EXAMPLE 4

### Antiviral assays and Cytotoxicity Assays

4.1. Material and Methods

4.1.1. Antiviral Assays

The herpes and vaccinia virus assays were based on inhibition of virus-induced cytopathicity in HEL cell cultures [herpes simplex virus type 1 (HSV-1) (KOS), HSV-2 (G), HSV-1 TK⁻ (KOS acyclovir resistant, ACV) and vaccinia virus (VV). Confluent cell cultures in microtiter 96-well plates were inoculated with 100 CCID₅₀ of virus (1 CCID₅₀ being the virus dose to infect 50% of the cell cultures) and the infected cell cultures were incubated in the presence of varying concentrations (200, 40, 8, . . . μM) of the test compounds. Viral cytopathogenicity was recorded as soon as it reached completion in the control virus-infected cell cultures that were not treated with the test compounds.

Confluent human embryonic lung (HEL) fibroblasts were grown in 96-well microtiter plates and infected with the human cytomegalovirus (HCMV) strains Davis and AD-169 at 100 PFU per well. After a 2-h incubation period, residual virus was removed and the infected cells were further incubated with medium containing different concentrations of the test compounds (in duplicate). After incubation for 7 days at 37° C., virus-induced cytopathogenicity was monitored microscopically after ethanol fixation and staining with Giemsa. Antiviral activity was expressed as the EC₅₀ or compound concentration required to reduce virus-induced cytopathogenicity by 50%. EC₅₀ values were calculated from graphic plots of the percentage of cytopathogenicity as a function of concentration of the compounds.

The laboratory wild-type VZV strain OKA and the thymidine kinase-deficient VZV strain 07/1 were used. Confluent HEL cell cultures grown in 96-well microtiter plates were inoculated with VZV at an input of 20 PFU per well. After a 2-h incubation period, residual virus was removed and varying concentrations of the test compounds were added (in duplicate). Antiviral activity was expressed as the 50%-effective concentration required to reduce viral plaque formation after 5 days by 50% as compared with untreated controls.

4.1.2. Cytotoxicity Assays

Cytotoxicity measurements were based on the inhibition of HEL cell growth. HEL cells were seeded at a rate of 5×10³ cells/well into 96-well microtiter plates and allowed to proliferate for 24 h. Then, medium containing different concentrations of the test compounds was added. After 3 days of incubation at 37° C., the cell number was determined with a Coulter counter. The 50%-cytostatic concentration (CC₅₀) was calculated as the compound concentration required to reduce cell growth by 50% relative to the number of cells in the untreated controls. CC₅₀ values were estimated from graphic plots of the number of cells (percentage of control) as a function of the concentration of the test compounds. Cytotoxicity was also expressed as the minimum cytotoxic concentration (MCC) or the compound concentration that causes a microscopically detectable alteration of cell morphology.

4.2. Results

They are given in FIG. 1.

The compounds were evaluated against a variety of DNA viruses including herpes simplex virus type 1 (HSV-1) (strain KOS), HSV-2 (strain G), thymidine kinase (TK)-deficient HSV-1 TK⁻, varicella-zoster virus (VZV) (strain OKA), the TK-deficient VZV TK⁻ (07/1), human cytomegalovirus (HCMV) and vaccinia virus (VV) in HEL cell cultures.

All derivatives according to the invention showed pronounced inhibitory activity against HSV-1, HSV-2 and VZV. In contrast with the [E] enantiomers (Ia1) and (Ie1), the corresponding [Z] enantiomers (Ia2 and Ie2) were clearly less, or not antivirally active.

The compounds were not significantly cytotoxic at 200 μM, but slightly cytostatic at 34-70 μM against HEL cell proliferation.

The [E] derivative (Iel) proved most inhibitory against HSV-1, HSV-2 and VZV. The independence of cellular TK activity is testified by the pronounced antiviral activity of the compound against mutant TK-deficient HSV-1 TK⁻ and VZV TK.

1. Compounds of formula (I) 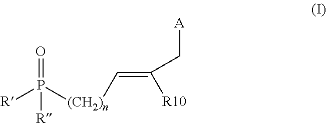
wherein
A represents a nucleobase or a derivative thereof,
n is equal to 0 or 1 and
R′ and R″ independently of each other
* represent a group selected from the group comprising
a hydroxy group with the proviso that R′ and R″ are not simultaneously a hydroxyl group,
O-methyl group,
O-benzyl group,
an oxymethylcarbonyl group of formula (2) 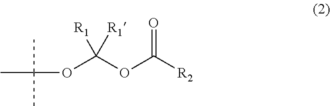
wherein
R₁ and R′₁ are independently of each other hydrogen or (C₁-C₄)alkyl group and
R₂ is a straight or branched (C₁-C₆)alkyl group or straight or branched (C₁-C₆)alkoxy group
a thioethylcarbonyl group of formula (3) 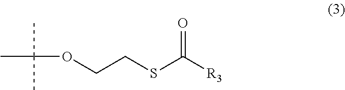
wherein
R₃ is a straight or branched (C₁-C₆)alkyl group
a lipohilic chain or
* R′ and R″ forms with the phosphate atom to which they are linked a cycloalkyle group of formula (4) 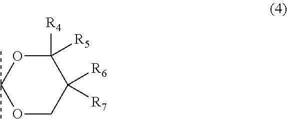
wherein R₄, R₅, R₆, et R₇ each independently represent a straight or branched (C₁-C₆)alkyl group or R₄, and R₇ independently represent a straight or branched (C₁-C₆)alkyl group and R₅ and R₆ form together an aromatic ring,
R₁₀ represents a hydrogen atom or a straight or branched (C₁-C₄)alkyl group optionally substituted by an OH group,
their diastereoisomers, or a pharmaceutically acceptable salt thereof,
for their use as antiviral agents, for the treatment of virus pathologies.

2. Compounds for their use in the treatment of pathologies due to viruses according to claim 1, said pathologies being selected from the group comprising Herpes virus, Vaccinia virus, Varicella-zoster virus, Cytomegalovirus, Vesicular stomatis virus, Influenza A, Coxsackie virus B4, respiratory syncytial virus, Feline corona virus, Feline herpes virus, Punta Toro Virus, HIV, Hepatitis B and Hepatitis C.

3. Compounds for their use as antiviral agents according to claim 2 wherein the virus is the Varicella-zoster virus with or without encoded thymidine kinase activity.

4. Compounds for their use as antiviral agents according to claim 1 wherein A is selected from the group comprising:
a) 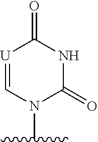
wherein U represents a nitrogen atom or C—R₁₁ with R₁₁ selected from the group comprising
a hydrogen atom,
an halogen atom selected from fluorine, chlorine, bromine and iodine,
a straight or branched (C₁-C₆)alkyl group
a 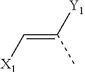
group with X₁ representing halogen atom and Y₁ representing a hydrogen atom or a halogen atom,
a phenyl group optionally substituted in the 4 position by a (C₁-C₆)alkyl group, a (C₁-C₆)alcoxy group, a phenyloxy, a 3,4-ethylenedioxy or a 3,4-methyledioxy,
a group selected from the group comprising thiophenyl, pyrrolyl or furanyl groups,
b) 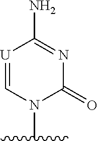
wherein U represents a nitrogen atom or C—R₁₆ with R₁₆ selected from the group comprising
a hydrogen atom, or
an halogen atom selected from fluorine, chlorine, bromine and iodine, 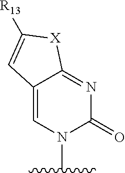
c)
Wherein
X represents an oxygen or a sulphur atom and R₁₃ represents a group selected from 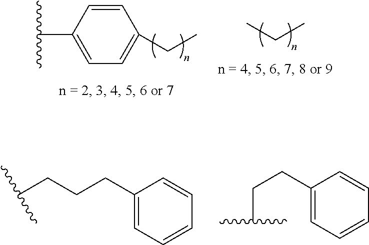 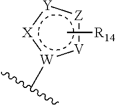
d)
wherein
V, W, X, Y, Z represents each independently of the other C or N, [symbol]
means a single or double bond according to the meaning of V, W, X, Y, Z and
R₁₄ is selected from the group comprising a hydrogen atom, a halogen atom, (C₁-C₄)alkyl groups, a phenyl group, ester groups, amide groups,
e) 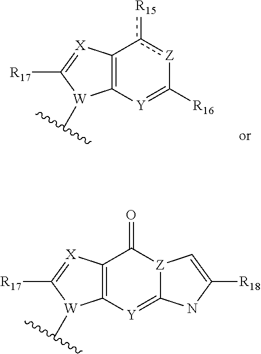
wherein
W, X, Y, Z represents each independently of the other C or N, [symbol]
means a single or double bond according to the meaning of W, X, Y, Z and R₁₅,
R₁₅ is selected from the group comprising
an halogen atom as defined above, advantageously a chlorine or a bromine,
an oxygen atom,
an amino group, or
a group selected from 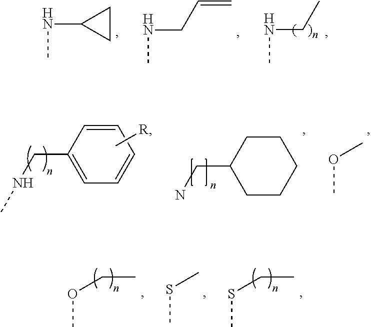
groups with R representing a straight or branched (C₁-C₄)alkyl group, preferably an isopropyl group and n being an integer equal to 0, 1, 2 or 3,
R₁₆ is a hydrogen atom or an amino group,
R₁₇ is selected from the group comprising a hydrogen atom, a halogen atom, (C₁-C₄)alkyl group and a phenyl group and
R₁₈ is selected from the group comprising a hydrogen atom, a halogen atom, (C₁-C₄)alkyl groups, ester groups and aromatic groups

5. Compounds for their use as antiviral agents according to claim 1 wherein R′ and R″ independently from each other are selected from the group comprising O-methyl, O-benzyl, or a biolabile group selected from the group comprising an oxymethylcarbonyl group or an alcoxyalkyl ester.

6. Compounds for their use as antiviral agents according to claim 1 wherein n is equal to 1.

7. Compounds for their use as antiviral agents according to claim 6 wherein R₁₀ is H and which are under the E form.

8. Pharmaceutical compositions comprising at least one compound according to claim 1 with a pharmaceutically acceptable carrier.

9. Pharmaceutical composition according to claim 8 further comprising an antiviral compounds selected from, but not limited to, the group comprising Abacavir, Acyclovir, Adefovir, Amprenavir, Atazanavir, Cidofovir, Darunavir, Delavirdine, Didanosine, Docosanol, Éfavirenz, Elvitégravir, Emtricitabine, Enfuvirtide, Étravirine, Famciclovir, Foscarnet, Fomivirsen, Ganciclovir, Indinavir, Idoxuridine, Lamivudine, Lopinavir Maraviroc, MK-2048, Nelfinavir, Nevirapine, Penciclovir, Raltégravir, Rilpivirine, Ritonavir, Saquinavir, Stavudine, Ténofovir Trifluridine, Valaciclovir, Valganciclovir, Vidarabine, Ibacitabine, Amantadine, Oseltamivir, Rimantidine, Tipranavir, Zalcitabine, Zanamivir et Zidovudine.

10. Compounds for their use as antiviral agents according to claim 2 wherein A is selected from the group comprising:
a) 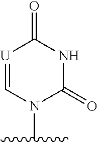
wherein U represents a nitrogen atom or C—R₁₁ with R₁₁ selected from the group comprising
a hydrogen atom,
an halogen atom selected from fluorine, chlorine, bromine and iodine,
a straight or branched (C₁-C₆)alkyl group
a 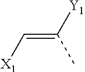
group with X₁ representing halogen atom and Y₁ representing a
hydrogen atom or a halogen atom,
a phenyl group optionally substituted in the 4 position by a (C₁-C₆)alkyl group, a (C₁-C₆)alcoxy group, a phenyloxy, a 3,4-ethylenedioxy or a 3,4-methyledioxy,
a group selected from the group comprising thiophenyl, pyrrolyl or furanyl groups,
b) 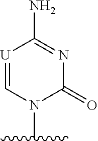
wherein U represents a nitrogen atom or C—R₁₆ with R₁₆ selected from the group comprising
a hydrogen atom, or
an halogen atom selected from fluorine, chlorine, bromine and iodine,
c) 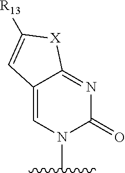
Wherein
X represents an oxygen or a sulphur atom and R₁₃ represents a group selected from 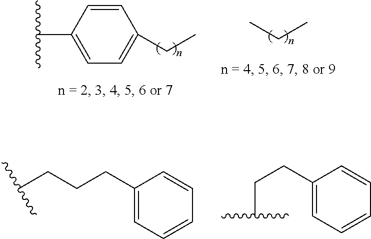
d) 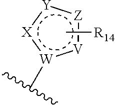
wherein
V, W, X, Y, Z represents each independently of the other C or N, [symbol]
means a single or double bond according to the meaning of V, W, X, Y, Z and
R₁₄ is selected from the group comprising a hydrogen atom, a halogen atom, (C₁-C₄)alkyl groups, a phenyl group, ester groups, amide groups,
e) 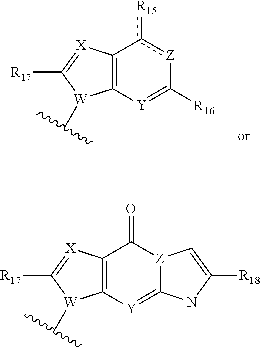
wherein
W, X, Y, Z represents each independently of the other C or N, [symbol]
means a single or double bond according to the meaning of W, X, Y, Z and R₁₅,
R₁₅ is selected from the group comprising
an halogen atom as defined above, advantageously a chlorine or a bromine,
an oxygen atom,
an amino group, or
a group selected from 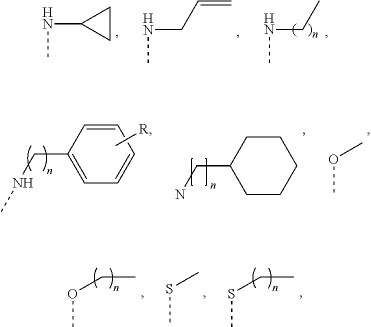
groups with R representing a straight or branched (C₁-C₄)alkyl group, preferably an isopropyl group and n being an integer equal to 0, 1, 2 or 3,
R₁₆ is a hydrogen atom or an amino group,
R₁₇ is selected from the group comprising a hydrogen atom, a halogen atom, (C₁-C₄)alkyl group and a phenyl group and
R₁₈ is selected from the group comprising a hydrogen atom, a halogen atom, (C₁-C₄)alkyl groups, ester groups and aromatic groups.

11. Compounds for their use as antiviral agents according to claim 3 wherein A is selected from the group comprising:
a) 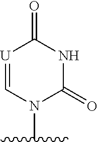
wherein U represents a nitrogen atom or C—R₁₁ with R₁₁ selected from the group comprising
a hydrogen atom,
an halogen atom selected from fluorine, chlorine, bromine and iodine,
a straight or branched (C₁-C₆)alkyl group
a 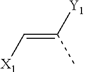
group with X₁ representing halogen atom and Y₁ representing a
hydrogen atom or a halogen atom,
a phenyl group optionally substituted in the 4 position by a (C₁-C₆)alkyl group, a (C₁-C₆)alcoxy group, a phenyloxy, a 3,4-ethylenedioxy or a 3,4-methyledioxy,
a group selected from the group comprising thiophenyl, pyrrolyl or furanyl groups,
b) 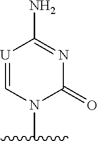
wherein U represents a nitrogen atom or C—R₁₆ with R₁₆ selected from the group comprising
a hydrogen atom, or
an halogen atom selected from fluorine, chlorine, bromine and iodine,
c) 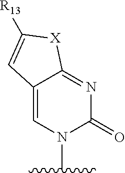
Wherein
X represents an oxygen or a sulphur atom and R₁₃ represents a group selected from 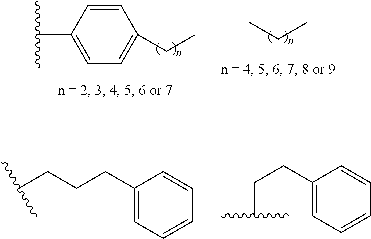
d) 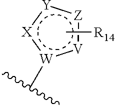
wherein
V, W, X, Y, Z represents each independently of the other C or N, [symbol]
means a single or double bond according to the meaning of V, W, X, Y, Z and
R₁₄ is selected from the group comprising a hydrogen atom, a halogen atom, (C₁-C₄)alkyl groups, a phenyl group, ester groups, amide groups,
e) 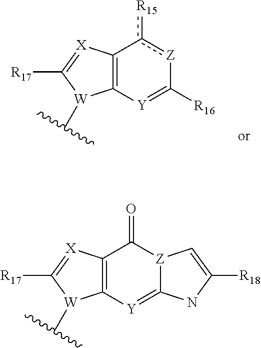
wherein
W, X, Y, Z represents each independently of the other C or N, [symbol]
means a single or double bond according to the meaning of W, X, Y, Z and R₁₅,
R₁₅ is selected from the group comprising
an halogen atom as defined above, advantageously a chlorine or a bromine,
an oxygen atom,
an amino group, or
a group selected from 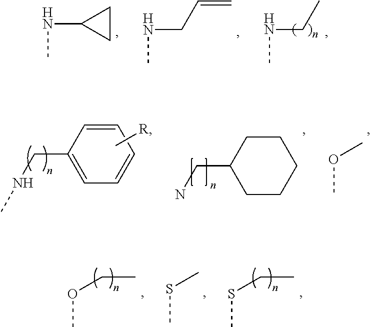
groups with R representing a straight or branched (C₁-C₄)alkyl group, preferably an isopropyl group and n being an integer equal to 0, 1, 2 or 3,
R₁₆ is a hydrogen atom or an amino group,
R₁₇ is selected from the group comprising a hydrogen atom, a halogen atom, (C₁-C₄)alkyl group and a phenyl group and
R₁₈ is selected from the group comprising a hydrogen atom, a halogen atom, (C₁-C₄)alkyl groups, ester groups and aromatic groups.

12. Compounds for their use as antiviral agents according to claim 2 wherein R′ and R″ independently from each other are selected from the group comprising O-methyl, O-benzyl, or a biolabile group selected from the group comprising an oxymethylcarbonyl group or an alcoxyalkyl ester.

13. Compounds for their use as antiviral agents according to claim 3 wherein R′ and R″ independently from each other are selected from the group comprising O-methyl, O-benzyl, or a biolabile group selected from the group comprising an oxymethylcarbonyl group or an alcoxyalkyl ester.

14. Compounds for their use as antiviral agents according to claim 4 wherein R′ and R″ independently from each other are selected from the group comprising O-methyl, O-benzyl, or a biolabile group selected from the group comprising an oxymethylcarbonyl group or an alcoxyalkyl ester.

15. Pharmaceutical compositions comprising at least one compound according to claim 2 with a pharmaceutically acceptable carrier.

16. Pharmaceutical compositions comprising at least one compound according to claim 3 with a pharmaceutically acceptable carrier.

17. Pharmaceutical compositions comprising at least one compound according to claim 4 with a pharmaceutically acceptable carrier.
